(19) United States
(12) Patent Application Publication
(10) Pub. No.: US 2022/0096780 A1
(43) Pub. Date: Mar. 31, 2022

(54) METHODS AND APPARATUS FOR TREATING A RESPIRATORY DISORDER

(71) Applicant: ResMed Pty Ltd, Bella Vista, NSW (AU)

(72) Inventors: Shayan MIARALIPOUR, Sydney (AU); Ronald James HUBY, North Epping (AU); Kiet Minh NGUYEN, Sidney (AU); David AGOSTINELLI, Melbourne, Victoria (AU)

(73) Assignee: ResMed Pty Ltd, Bella Vista, NSW (AU)

(21) Appl. No.: 17/422,582

(22) PCT Filed: Feb. 4, 2020
(86) PCT No.: PCT/AU2020/050074

§ 371 Date: Jul. 13, 2021

Related U.S. Application Data
(60) Provisional application No. 62/802,076, filed on Feb. 6, 2019.

(51) Int. Cl. A61M 16/10; B01D 53/047; A61M 16/00

(52) U.S. Cl. CPC A61M 16/101; A61M 2205/3368; A61M 16/026; A61M 2205/3331; A61M 2205/52; B01D 2259/40009; A61M 2205/3334; A61M 2205/3553; A61M 2205/502; B01D 2259/40011; A61M 2205/3379; A61M 2205/3327; B01D 2253/108; B01D 2259/4533; B01D 2257/102; B01D 53/047

(57) ABSTRACT

Apparatus, such as a portable oxygen concentrator (100) or other device communicating therewith, may be configured, such as with a processor(s), to estimate a remaining capacity of a sieve bed of the concentrator. Such apparatus may be configured to access a parameter of a measured pressure-time characteristic of the sieve bed for a phase of a pressure swing adsorption cycle of the oxygen concentrator. The apparatus may be configured to access function(s) of the parameter of the pressure-time characteristic and operational characteristic(s) of the sieve bed. The apparatus may be configured to estimate the remaining capacity by applying the function(s) to the parameter of the measured pressure-time characteristic. Such an estimate may then serve as a basis for providing notification, such as on a display or by electronic messaging, to inform of remaining life of the sieve bed, or otherwise promote timely replacement of a depleting component.

### CROSS-REFERENCE TO RELATED APPLICATIONS

This application claims the benefit of U.S. Provisional Application No. 62/802,076, filed 6 Feb. 2019, the entire disclosure of which are hereby incorporated herein by reference.

### FIELD OF THE TECHNOLOGY

The present technology relates generally to methods and apparatus for treating respiratory disorders, and more specifically to methods and apparatus for estimating the remaining life/capacity of sieve beds used by an oxygen concentrator for supplying oxygen enriched gas to patients with respiratory disorders.

### DESCRIPTION OF THE RELATED ART

There are many users that require supplemental oxygen as part of Long Term Oxygen Therapy (LTOT). Currently, the vast majority of users that are receiving LTOT are diagnosed under the general category of Chronic Obstructive Pulmonary Disease (COPD). This general diagnosis includes such common diseases as Chronic Asthma, Emphysema, and several other cardio-pulmonary conditions. Other users may also require supplemental oxygen, for example, obese individuals to maintain elevated activity levels, or infants with cystic fibrosis or broncho-pulmonary dysplasia.

Doctors may prescribe oxygen concentrators or portable tanks of medical oxygen for these users. Usually a specific continuous oxygen flow rate is prescribed (e.g., 1 litre per minute (LPM), 2 LPM, 3 LPM, etc.). Experts in this field have also recognized that exercise for these users provide long term benefits that slow the progression of the disease, improve quality of life and extend user longevity. Most stationary forms of exercise like tread mills and stationary bicycles, however, are too strenuous for these users. As a result, the need for mobility has long been recognized. Until recently, this mobility has been facilitated by the use of small compressed oxygen tanks. The disadvantage of these tanks is that they have a finite amount of oxygen and they are heavy, weighing about 50 pounds, when mounted on a cart with dolly wheels.

Oxygen concentrators have been in use for about 50 years to supply users suffering from respiratory insufficiency with supplemental oxygen with oxygen enriched gas. Traditional oxygen concentrators used to provide these flow rates have been bulky and heavy making ordinary ambulatory activities with them difficult and impractical. Recently, companies that manufacture large stationary home oxygen concentrators began developing portable oxygen concentrators (POCs). The advantage of POCs is that they can produce a theoretically endless supply of oxygen enriched gas. In order to make these devices small for mobility, the various systems necessary for the production of oxygen enriched gas are condensed.

Oxygen concentrators may take advantage of pressure swing adsorption (PSA). Pressure swing adsorption involves using a compressor to increase gas pressure inside a canister that contains particles of a gas separation adsorbent that attracts nitrogen more strongly than it does oxygen. The mass of adsorbent particles inside the canister is referred to as a sieve bed. Ambient air usually includes approximately 78% nitrogen and 21% oxygen with the balance comprised of argon, carbon dioxide, water vapor and other trace gases. If a feed gas mixture such as air, for example, is passed under pressure through a sieve bed, part or all of the nitrogen will be adsorbed by the sieve bed, and the gas coming out of the vessel will be enriched in oxygen. When the sieve bed reaches the end of its capacity to adsorb nitrogen, it can be regenerated by reducing the pressure, thereby releasing the adsorbed nitrogen. It is then ready for another “PSA cycle” of producing oxygen enriched gas. By alternating canisters in a two-canister system, one canister can be concentrating oxygen (the so-called “adsorption phase”) while the other canister is being purged (the “purge phase”). This alternation results in a near-continuous separation of the oxygen from the nitrogen. In this manner, oxygen can be concentrated out of the air for a variety of needs, including providing supplemental oxygen to users. Further details regarding oxygen concentrators may be found, for example, in U.S. Published Patent Application No. 2009-0065007, published Mar. 12, 2009, and entitled “Oxygen Concentrator Apparatus and Method”, which is incorporated herein by reference.

The gas separation adsorbents used in POCs have a very high affinity for water. This affinity is so high that it overcomes nitrogen affinity, and thus when both water vapor and nitrogen are available in a feed gas stream (such as ambient air), the adsorbent will preferentially adsorb water vapor over nitrogen. Furthermore when it is adsorbed, water does not desorb as easily as nitrogen. As a result, water molecules remain adsorbed even after regeneration and thus block the adsorption sites for nitrogen. Therefore over time and use, water accumulates on the adsorbent, which becomes less and less efficient for nitrogen adsorption, to the point where the sieve bed needs to be replaced because little further oxygen concentration can take place. Such sieve beds may be referred to as exhausted or deactivated.

It would be advantageous for a POC to be able to estimate the remaining nitrogen-adsorbing capacity of its sieve bed(s). Users could then be kept informed of the remaining capacity in order to plan for replacement of an exhausted sieve bed or a sieve bed approaching exhaustion giving unacceptable performance.

### SUMMARY

Disclosed herein are methods and apparatus for estimating the remaining capacity of a sieve bed in a portable oxygen concentrator. As a sieve bed becomes deactivated, less nitrogen is adsorbed by the adsorbent, and therefore more of the nitrogen is available to increase the pressure inside the sieve bed. The pressure therefore increases more rapidly as the feed gas is fed in. Analysis of the sieve bed pressure-time characteristic over the PSA cycle thus allows estimation of the remaining adsorption capacity of the adsorbent material in the sieve bed. The disclosed methods and apparatus extract one or more parameters of the pressure-time characteristic and base an estimate of the remaining capacity of the sieve beds on the one or more parameters.

Some versions of the present technology include a method of estimating a remaining capacity of a sieve bed in an oxygen concentrator. The method may include accessing a parameter of a measured pressure-time characteristic of the sieve bed for a phase of a pressure swing adsorption cycle of the oxygen concentrator. The method may include accessing one or more functions of the parameter of the measured pressure-time characteristic and one or more operational characteristic(s) of the sieve bed. The method may include estimating the remaining capacity by applying the one or more functions to the parameter of the measured pressure-time characteristic.

In some versions, the one or more functions may use a fresh value of the parameter for a fresh sieve bed of the same type as the sieve bed. The method may include adjusting at least one of (a) the fresh value of the parameter, and (2) the parameter, to compensate for a change in ambient conditions since the fresh value of the parameter was measured. The one or more functions may use an exhausted value of the parameter for an exhausted sieve bed of the same type as the sieve bed. The method may include adjusting the exhausted value of the parameter to compensate for a change in ambient conditions since the exhausted value of the parameter was measured. The one or more functions may include an interpolation, such as linear interpolation, using the fresh value of the parameter and the exhausted value of the parameter. The parameter may be a pressure rise time. The parameter may be an unadsorbed fraction of feed gas fed into the sieve bed. The one or more functions may include a parameter representing a total number of moles of feed gas fed into the sieve bed. The one or more functions may include a parameter representing a mass flow rate of feed gas fed into the sieve bed. The one or more functions may include a parameter representing an amount of unadsorbed feed gas fed into the sieve bed that have increased a pressure of a canister of the sieve bed. The one or more functions may include parameters representing a change in pressure over the phase, a void volume of a canister of the sieve bed, a temperature of feed gas fed into the sieve bed, and a universal gas constant. The one or more functions may include dividing an amount of unadsorbed feed gas fed into the sieve bed by an amount of feed gas fed into the sieve bed.

In some versions, the one or more functions may include one or more look-up tables. The method may further include repeating the accessing and the estimating to obtain a further estimate of remaining capacity. The method may further include estimating a remaining usage time of the sieve bed from the estimate and the further estimate of remaining capacity. The method may further include displaying, on a display of the oxygen concentrator, an indicator of the estimate of remaining capacity. The method may further include generating a message based on the estimate of remaining capacity.

In some versions, the method may include measuring the pressure-time characteristic of the sieve bed for the phase of the pressure swing adsorption cycle of the oxygen concentrator to generate the parameter. The method may include repeating the measuring to obtain a further estimate of remaining capacity.

Some versions of the present technology may include a method of estimating a remaining capacity of a sieve bed in an oxygen concentrator. The method may include measuring a pressure-time characteristic of the sieve bed for a phase of a pressure swing adsorption cycle of the oxygen concentrator. The method may include extracting a parameter of the pressure-time characteristic. The method may include estimating the remaining capacity using the parameter of the pressure-time characteristic.

In some versions, the parameter is an unadsorbed fraction of feed gas fed into the sieve bed. In some versions, the parameter is a pressure rise time. The method may further include e repeating the measuring, extracting, and estimating to obtain a further estimate of remaining capacity. The method may include estimating remaining usage time of the sieve bed from the estimate and the further estimate of remaining capacity.

Some versions of the present technology may include an oxygen concentrator. The oxygen concentrator may include a sieve bed containing a gas separation adsorbent. The oxygen concentrator may include a compression system configured to feed a feed gas into the sieve bed. The oxygen concentrator may include a memory and a controller. The controller may include one or more processors. The one or more processors may be configured by program instructions stored in the memory to execute a method of estimating a remaining capacity of the sieve bed such as a method of the methods previously described or further described herein.

Some versions of the present technology may include an oxygen concentrator. The oxygen concentrator may include a sieve bed containing a gas separation adsorbent. The oxygen concentrator may include a compression system configured to feed a feed gas into the sieve bed. The oxygen concentrator may include a memory and a controller. The controller may be configured to access a parameter of a measured pressure-time characteristic of the sieve bed for a phase of a pressure swing adsorption cycle of the oxygen concentrator. The controller may be configured to access one or more functions of the parameter of the pressure-time characteristic and one or more operational characteristics of the sieve bed. The controller may be configured to estimate the remaining capacity by applying the one or more functions to the parameter of the measured pressure-time characteristic.

Some versions of the present technology may include an oxygen concentrator. The oxygen concentrator may include a sieve bed containing a gas separation adsorbent. The oxygen concentrator may include a compression system configured to feed a feed gas into the sieve bed. The oxygen concentrator may include a memory. The oxygen concentrator may include a controller. The controller may be configured to measure, such as with one or more sensors, a pressure-time characteristic of the sieve bed for a phase of a pressure swing adsorption cycle of the oxygen concentrator. The controller may be configured to extract a parameter of the pressure-time characteristic. The controller may be configured to estimate the remaining capacity using the parameter of the pressure-time characteristic.

Some versions of the present technology may include a connected oxygen therapy system. The system may include a portable oxygen concentrator that may include a sieve bed containing a gas separation adsorbent. The system may include an external computing device in communication with the portable oxygen concentrator. The system may include a memory and a processor configured by program instructions stored in the memory to execute a method of estimating a remaining capacity of the sieve bed such as any described herein. Such a method may include accessing a parameter of a measured pressure-time characteristic of the sieve bed for a phase of a pressure swing adsorption cycle of the portable oxygen concentrator. Such a method may include accessing one or more functions of the parameter of the pressure-time characteristic and one or more operational characteristics of the sieve bed. Such a method may include estimating the remaining capacity by applying the one or more functions to the parameter of the measured pressure-time characteristic.

In some versions, the processor and the memory may be part of the portable oxygen concentrator. The processor may be further configured to transmit the remaining capacity estimate to the external computing device. The processor and the memory may be part of the external computing device. The system may include a display. The processor may be further configured to display an indicator of the estimate of remaining capacity on the display. The external computing device may be a portable computing device. The external computing device may be a server. The system further may include a personal computing device in communication with the server. The personal computing device may be configured to interact with a portal system hosted by the server. The personal computing device may be configured to receive the remaining capacity estimate from the portal system. The personal computing device may be configured to display the remaining capacity estimate on a display of the personal computing device.

In some versions, the system may further include a portable computing device in communication with the server. The portable computing device may be configured to receive the remaining capacity estimate from the server. The portable computing device may be configured to display the remaining capacity estimate on a display of the portable computing device.

Some versions of the present technology may include apparatus that may include means for accessing a parameter of a measured pressure-time characteristic of a sieve bed for a phase of a pressure swing adsorption cycle of an oxygen concentrator. The apparatus may include means for accessing one or more functions of the parameter of the pressure-time characteristic and one or more operational characteristics of the sieve bed. The apparatus may include means for estimating a remaining capacity of the sieve bed by applying the one or more functions to the parameter of the measured pressure-time characteristic.

Some versions of the present technology may include apparatus including means for measuring a pressure-time characteristic of a sieve bed for a phase of a pressure swing adsorption cycle of an oxygen concentrator. The apparatus may include means for extracting a parameter of the pressure-time characteristic. The apparatus may include means for estimating a remaining capacity of the a sieve bed using the parameter of the pressure-time characteristic.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### BRIEF DESCRIPTION OF THE DRAWINGS

Advantages of the present technology will become apparent to those skilled in the art with the benefit of the following detailed description of embodiments and upon reference to the accompanying drawings in which:

FIG. 1 depicts a schematic diagram of the components of an oxygen concentrator;

FIG. 2 depicts a side view of examples of main components of an oxygen concentrator;

FIG. 3A depicts a perspective side view of a compression system;

FIG. 3B depicts a side perspective view of a compression system that includes a heat exchange conduit;

FIG. 4A depicts a schematic diagram of the outlet components of an oxygen concentrator;

FIG. 4B depicts an outlet conduit for an oxygen concentrator;

FIG. 4C depicts an alternate outlet conduit for an oxygen concentrator;

FIG. 5 depicts an outer housing for an oxygen concentrator;

FIG. 6 depicts a control panel for an oxygen concentrator;

FIG. 7 a communication arrangement of example devices that may be implemented in a connected oxygen therapy system 50;

FIG. 8 is a graph illustrating the linear interpolation of remaining capacity from the computed fraction based on the “fresh fraction”.

FIG. 9 is a graph illustrating the linear interpolation of remaining capacity from a measured pressure rise time based on the “fresh” pressure rise time and the “exhausted” pressure rise time.

FIG. 10 contains a flowchart of a method of estimating the remaining capacity of a sieve bed in one implementation of the present technology.

FIG. 11 contains a flowchart of a method of estimating the remaining capacity of a sieve bed in one implementation of the present technology.

While the technology is susceptible to various modifications and alternative forms, specific embodiments thereof are shown by way of example in the drawings and will herein be described in detail. It should be understood, however, that the drawings and detailed description thereto are not intended to limit the technology to the particular form disclosed, but on the contrary, the intention is to cover all modifications, equivalents, and alternatives falling within the spirit and scope of the present technology as defined by the appended claims.

### DETAILED DESCRIPTION OF THE EMBODIMENTS

It is to be understood the present technology is not limited to particular devices or methods, which may, of course, vary. It is also to be understood that the terminology used herein is for the purpose of describing particular embodiments only, and is not intended to be limiting. Headings are for organizational purposes only and are not meant to be used to limit or interpret the description or claims. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.”

The term “coupled” as used herein means either a direct connection or an indirect connection (e.g., one or more intervening connections) between one or more objects or components. The phrase “connected” means a direct connection between objects or components such that the objects or components are connected directly to each other. As used herein the phrase “obtaining” a device means that the device is either purchased or constructed.

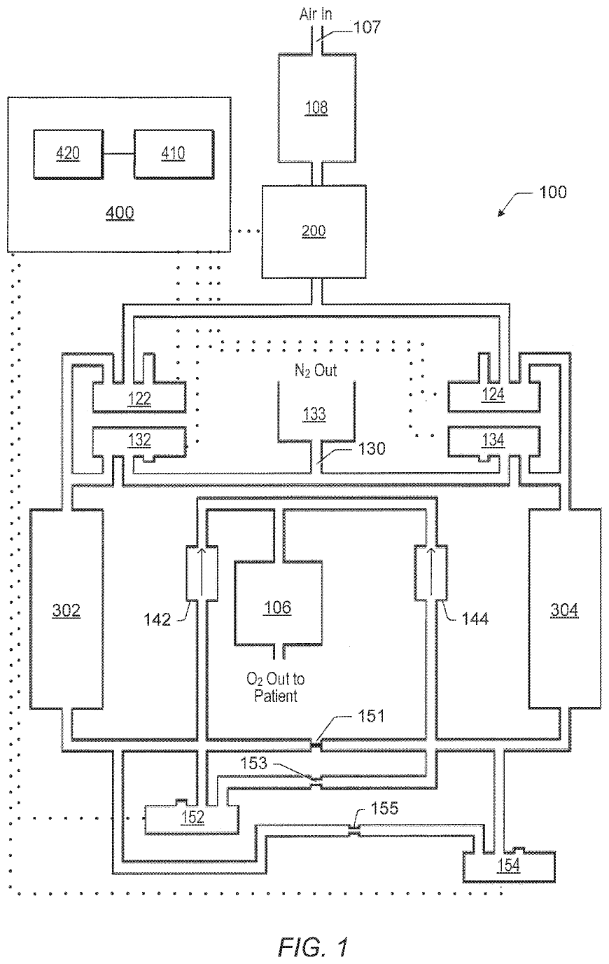

FIG. 1 illustrates a schematic diagram of an oxygen concentrator 100, according to an embodiment. Oxygen concentrator 100 may concentrate oxygen out of an air stream to provide oxygen enriched gas to a user. As used herein, “oxygen enriched gas” is composed of at least about 50% oxygen, at least about 60% oxygen, at least about 70% oxygen, at least about 80% oxygen, at least about 90% oxygen, at least about 95% oxygen, at least about 98% oxygen, or at least about 99% oxygen.

Oxygen concentrator 100 may be a portable oxygen concentrator. For example, oxygen concentrator 100 may have a weight and size that allows the oxygen concentrator to be carried by hand and/or in a carrying case. In one embodiment, oxygen concentrator 100 has a weight of less than about 20 lbs, less than about 15 lbs, less than about 10 lbs, or less than about 5 lbs. In an embodiment, oxygen concentrator 100 has a volume of less than about 1000 cubic inches, less than about 750 cubic inches; less than about 500 cubic inches, less than about 250 cubic inches, or less than about 200 cubic inches.

### Canisters

Oxygen may be collected from a feed gas by pressurising the feed gas in canisters 302 and 304, which contain a gas separation adsorbent. Gas separation adsorbents useful in an oxygen concentrator are capable of separating at least nitrogen from an air stream to leave oxygen enriched gas. Examples of gas separation adsorbents include molecular sieves that are capable of separation of nitrogen from an air stream. Examples of adsorbents that may be used in an oxygen concentrator include, but are not limited to, zeolites (natural) or synthetic crystalline aluminosilicates that separate nitrogen from oxygen in an air stream under elevated pressure. Examples of synthetic crystalline aluminosilicates that may be used include, but are not limited to: OXYSIV adsorbents available from UOP LLC, Des Plaines, IW; SYLOBEAD adsorbents available from W. R. Grace & Co, Columbia, Md.; SILIPORITE adsorbents available from CECA S.A. of Paris, France; ZEOCHEM adsorbents available from Zeochem AG, Uetikon, Switzerland; and AgLiLSX adsorbent available from Air Products and Chemicals, Inc., Allentown, Pa.

As shown in FIG. 1, air may enter the oxygen concentrator through air inlet 107. Air may be drawn into air inlet 107 by compression system 200. Compression system 200 may draw in air from the surroundings of the oxygen concentrator and compress the air, forcing the compressed air into one or both canisters 302 and 304. In an embodiment, an inlet muffler 108 may be coupled to air inlet 107 to reduce sound produced by air being pulled into the oxygen concentrator by compression system 200. In an embodiment, inlet muffler 108 may be a moisture and sound absorbing muffler. For example, a water absorbent material (such as a polymer water absorbent material or a zeolite material) may be used to both absorb water from the incoming air and to reduce the sound of the air passing into the air inlet 107.

Compression system 200 may include one or more compressors capable of compressing air. Pressurized air, produced by compression system 200, may be forced into one or both of the canisters 302 and 304. In some embodiments, the feed gas may be pressurized in the canisters to a pressure approximately in a range of up to 30 pounds per square inch (psi). Other pressures may also be used, depending on the type of gas separation adsorbent disposed in the canisters.

Coupled to each canister 302/304 are inlet valves 122/124 and outlet valves 132/134. As shown in FIG. 1, inlet valve 122 is coupled to canister 302 and inlet valve 124 is coupled to canister 304. Outlet valve 132 is coupled to canister 302 and outlet valve 134 is coupled to canister 304. Inlet valves 122/124 are used to control the passage of air from compression system 200 to the respective canisters. Outlet valves 132/134 are used to release gas from the respective canisters during a venting process. In some embodiments, inlet valves 122/124 and outlet valves 132/134 may be silicon plunger solenoid valves. Other types of valves, however, may be used. Plunger valves offer advantages over other kinds of valves by being quiet and having low leakage.

In some embodiments, a two-step valve actuation voltage may be used to control inlet valves 122/124 and outlet valves 132/134. For example, a high voltage (e.g., 24 V) may be applied to an inlet valve to open the inlet valve. The voltage may then be reduced (e.g., to 7 V) to keep the inlet valve open. Using less voltage to keep a valve open may use less power (Power=Voltage*Current). This reduction in voltage minimizes heat buildup and power consumption to extend run time from the power supply 180 (described below). When the power is cut off to the valve, it closes by spring action. In some embodiments, the voltage may be applied as a function of time that is not necessarily a stepped response (e.g., a curved downward voltage between an initial 24 V and a final 7 V).

In an embodiment, pressurized air is fed into one of canisters 302 or 304 while the other canister is being depressurized. For example, during use, inlet valve 122 is opened while inlet valve 124 is closed. Pressurized air from compression system 200 is forced into canister 302, while being inhibited from entering canister 304 by inlet valve 124. In an embodiment, a controller 400 is electrically coupled to valves 122, 124, 132, and 134. Controller 400 includes one or more processors 410 operable to execute program instructions stored in memory 420. The program instructions are operable to perform various predefined methods that are used to operate the oxygen concentrator. Controller 400 may include program instructions for operating inlet valves 122 and 124 out of phase with each other, i.e., when one of inlet valves 122 or 124 is opened, the other valve is closed. During pressurization of canister 302, outlet valve 132 is closed and outlet valve 134 is opened. Similar to the inlet valves, outlet valves 132 and 134 are operated out of phase with each other. In some embodiments, the voltages and the duration of the voltages used to open the input and output valves may be controlled by controller 400.

Check valves 142 and 144 are coupled to canisters 302 and 304, respectively. Check valves 142 and 144 are one way valves that are passively operated by the pressure differentials that occur as the canisters are pressurized and vented. Check valves 142 and 144 are coupled to canisters to allow oxygen enriched gas produced during pressurization of the canister to flow out of the canister, and to inhibit back flow of oxygen enriched gas or any other gases into the canister. In this manner, check valves 142 and 144 act as one way valves allowing oxygen enriched gas to exit the respective canister while pressurized.

The term “check valve”, as used herein, refers to a valve that allows flow of a fluid (gas or liquid) in one direction and inhibits back flow of the fluid. Examples of check valves that are suitable for use include, but are not limited to: a ball check valve; a diaphragm check valve; a butterfly check valve; a swing check valve; a duckbill valve; and a lift check valve. Under pressure, nitrogen molecules in the pressurized feed gas are adsorbed by the gas separation adsorbent in the pressurized canister. As the pressure increases, more nitrogen is adsorbed until the gas in the canister is enriched in oxygen. The nonadsorbed gas molecules (mainly oxygen) flow out of the pressurized canister when the pressure difference across the check valve coupled to the canister reaches a value sufficient to overcome the resistance of the check valve. In one embodiment, the pressure drop of the check valve in the forward direction is less than 1 psi. The break pressure in the reverse direction is greater than 100 psi. It should be understood, however, that modification of one or more components would alter the operating parameters of these valves. If the forward flow pressure is increased, there is, generally, a reduction in oxygen enriched gas production. If the break pressure for reverse flow is reduced or set too low, there is, generally, a reduction in oxygen enriched gas pressure.

In an exemplary embodiment, canister 302 is pressurized by compressed air produced in compression system 200 and passed into canister 302. During pressurization of canister 302 inlet valve 122 is open, outlet valve 132 is closed, inlet valve 124 is closed and outlet valve 134 is open. Outlet valve 134 is opened when outlet valve 132 is closed to allow substantially simultaneous venting of canister 304 while canister 302 is pressurized. Canister 302 is pressurized until the pressure in canister is sufficient to open check valve 142. Oxygen enriched gas produced in canister 302 exits through check valve and, in one embodiment, is collected in accumulator 106.

After some time the gas separation adsorbent will become saturated with nitrogen and will be unable to separate significant amounts of nitrogen from incoming air. In the embodiment described above, when the gas separation adsorbent in canister 302 reaches this saturation point, the inflow of compressed air is stopped and canister 302 is vented to remove nitrogen. During venting, inlet valve 122 is closed, and outlet valve 132 is opened. While canister 302 is being vented, canister 304 is pressurized to produce oxygen enriched gas in the same manner described above. Pressurization of canister 304 is achieved by closing outlet valve 134 and opening inlet valve 124. The oxygen enriched gas exits canister 304 through check valve 144.

During venting of canister 302, outlet valve 132 is opened allowing pressurized gas (mainly nitrogen) to exit the canister through concentrator outlet 130. In an embodiment, the vented gases may be directed through muffler 133 to reduce the noise produced by releasing the pressurized gas from the canister. As gas is released from canister 302, the pressure in the canister drops, allowing the nitrogen to become desorbed from the gas separation adsorbent. The released nitrogen exits the canister through outlet 130, resetting the canister to a state that allows renewed separation of oxygen from an air stream. Muffler 133 may include open cell foam (or another material) to muffle the sound of the gas leaving the oxygen concentrator. In some embodiments, the combined muffling components/techniques for the input of air and the output of gas, may provide for oxygen concentrator operation at a sound level below 50 decibels.

During venting of the canisters, it is advantageous that at least a majority of the nitrogen is removed. In an embodiment, at least about 50%, at least about 60%, at least about 70%, at least about 80%, at least about 90%, at least about 95%, at least about 98%, or substantially all of the nitrogen in a canister is removed before the canister is re-used to separate oxygen from air. In some embodiments, a canister may be further purged of nitrogen using an oxygen enriched stream that is introduced into the canister from the other canister.

In an exemplary embodiment, a portion of the oxygen enriched gas may be transferred from canister 302 to canister 304 when canister 304 is being vented of nitrogen. Transfer of oxygen enriched gas from canister 302 to 304, during venting of canister 304, helps to further purge nitrogen (and other gases) from the canister. In an embodiment, oxygen enriched gas may travel through flow restrictors 151, 153, and 155 between the two canisters. Flow restrictor 151 may be a trickle flow restrictor. Flow restrictor 151, for example, may be a 0.009 D flow restrictor (e.g., the flow restrictor has a radius 0.009″ which is less than the diameter of the tube it is inside). Flow restrictors 153 and 155 may be 0.013 D flow restrictors. Other flow restrictor types and sizes are also contemplated and may be used depending on the specific configuration and tubing used to couple the canisters. In some embodiments, the flow restrictors may be press fit flow restrictors that restrict air flow by introducing a narrower diameter in their respective tube. In some embodiments, the press fit flow restrictors may be made of sapphire, metal or plastic (other materials are also contemplated).

Flow of oxygen enriched gas is also controlled by use of valve 152 and valve 154. Valves 152 and 154 may be opened for a short duration during the venting process (and may be closed otherwise) to prevent excessive oxygen loss out of the purging canister. Other durations are also contemplated. In an exemplary embodiment, canister 302 is being vented and it is desirable to purge canister 302 by passing a portion of the oxygen enriched gas being produced in canister 304 into canister 302. A portion of oxygen enriched gas, upon pressurization of canister 304, will pass through flow restrictor 151 into canister 302 during venting of canister 302. Additional oxygen enriched gas is passed into canister 302, from canister 304, through valve 154 and flow restrictor 155. Valve 152 may remain closed during the transfer process, or may be opened if additional oxygen enriched gas is needed. The selection of appropriate flow restrictors 151 and 155, coupled with controlled opening of valve 154 allows a controlled amount of oxygen enriched gas to be sent from canister 304 to 302. In an embodiment, the controlled amount of oxygen enriched gas is an amount sufficient to purge canister 302 and minimize the loss of oxygen enriched gas through venting valve 132 of canister 302. While this embodiment describes venting of canister 302, it should be understood that the same process can be used to vent canister 304 using flow restrictor 151, valve 152 and flow restrictor 153.

The pair of equalization/vent valves 152/154 work with flow restrictors 153 and 155 to optimize the air flow balance between the two canisters. This may allow for better flow control for venting the canisters with oxygen enriched gas from the other of the canisters. It may also provide better flow direction between the two canisters. It has been found that, while flow valves 152/154 may be operated as bi-directional valves, the flow rate through such valves varies depending on the direction of fluid flowing through the valve. For example, oxygen enriched gas flowing from canister 304 toward canister 302 has a flow rate faster through valve 152 than the flow rate of oxygen enriched gas flowing from canister 302 toward canister 304 through valve 152. If a single valve was to be used, eventually either too much or too little oxygen enriched gas would be sent between the canisters and the canisters would, over time, begin to produce different amounts of oxygen enriched gas. Use of opposing valves and flow restrictors on parallel air pathways may equalize the flow pattern of the oxygen between the two canisters. Equalising the flow may allow for a steady amount of oxygen to be available to the user over multiple cycles and also may allow a predictable volume of oxygen to purge the other of the canisters. In some embodiments, the air pathway may not have restrictors but may instead have a valve with a built in resistance or the air pathway itself may have a narrow radius to provide resistance.

At times, oxygen concentrator may be shut down for a period of time. When an oxygen concentrator is shut down, the temperature inside the canisters may drop as a result of the loss of adiabatic heat from the compression system. As the temperature drops, the volume occupied by the gases inside the canisters will drop. Cooling of the canisters may lead to a negative pressure in the canisters. Valves (e.g., valves 122, 124, 132, and 134) leading to and from the canisters are dynamically sealed rather than hermetically sealed. Thus, outside air may enter the canisters after shutdown to accommodate the pressure differential. When outside air enters the canisters, moisture from the outside air may condense inside the canister as the air cools. Condensation of water inside the canisters may lead to gradual degradation of the gas separation adsorbents, steadily reducing ability of the gas separation adsorbents to produce oxygen enriched gas.

In an embodiment, outside air may be inhibited from entering canisters after the oxygen concentrator is shut down by pressurising both canisters prior to shutdown. By storing the canisters under a positive pressure, the valves may be forced into a hermetically closed position by the internal pressure of the air in the canisters. In an embodiment, the pressure in the canisters, at shutdown, should be at least greater than ambient pressure. As used herein the term “ambient pressure” refers to the pressure of the surroundings in which the oxygen concentrator is located (e.g. the pressure inside a room, outside, in a plane, etc.). In an embodiment, the pressure in the canisters, at shutdown, is at least greater than standard atmospheric pressure (i.e., greater than 760 mmHg (Torr), 1 atm, 101,325 Pa). In an embodiment, the pressure in the canisters, at shutdown, is at least about 1.1 times greater than ambient pressure; is at least about 1.5 times greater than ambient pressure; or is at least about 2 times greater than ambient pressure.

In an embodiment, pressurization of the canisters may be achieved by directing pressurized air into each canister from the compression system and closing all valves to trap the pressurized air in the canisters. In an exemplary embodiment, when a shutdown sequence is initiated, inlet valves 122 and 124 are opened and outlet valves 132 and 134 are closed. Because inlet valves 122 and 124 are joined together by a common conduit, both canisters 302 and 304 may become pressurized as air and or oxygen enriched gas from one canister may be transferred to the other canister. This situation may occur when the pathway between the compression system and the two inlet valves allows such transfer. Because the oxygen concentrator operates in an alternating pressurize/venting mode, at least one of the canisters should be in a pressurized state at any given time. In an alternate embodiment, the pressure may be increased in each canister by operation of compression system 200. When inlet valves 122 and 124 are opened, pressure between canisters 302 and 304 will equalize, however, the equalized pressure in either canister may not be sufficient to inhibit air from entering the canisters during shutdown. In order to ensure that air is inhibited from entering the canisters, compression system 200 may be operated for a time sufficient to increase the pressure inside both canisters to a level at least greater than ambient pressure. Regardless of the method of pressurization of the canisters, once the canisters are pressurized, inlet valves 122 and 124 are closed, trapping the pressurized air inside the canisters, which inhibits air from entering the canisters during the shutdown period.

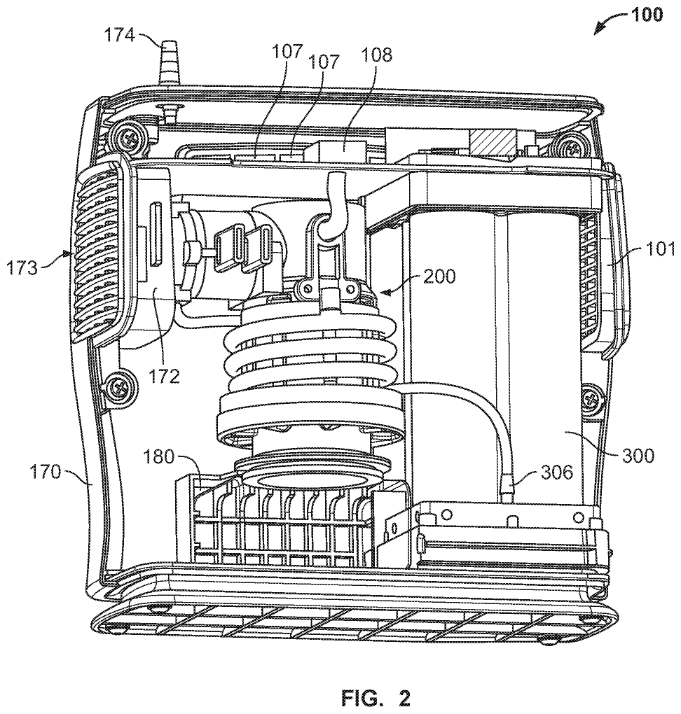

Referring to FIG. 2, an embodiment of an oxygen concentrator 100 is depicted. Oxygen concentrator 100 includes a compression system 200, a canister assembly 300, and a power supply 180 disposed within an outer housing 170. Inlets 101 are located in outer housing 170 to allow air from the environment to enter oxygen concentrator 100. Inlets 101 may allow air to flow into the compartment to assist with cooling of the components in the compartment. Power supply 180 provides a source of power for the oxygen concentrator 100. Compression system 200 draws air in through the inlet 107 and muffler 108. Muffler 108 may reduce noise of air being drawn in by the compression system and also may include a desiccant material to remove water vapour from the incoming air. Oxygen concentrator 100 may further include fan 172 used to vent air and other gases from the oxygen concentrator.

### Compression System

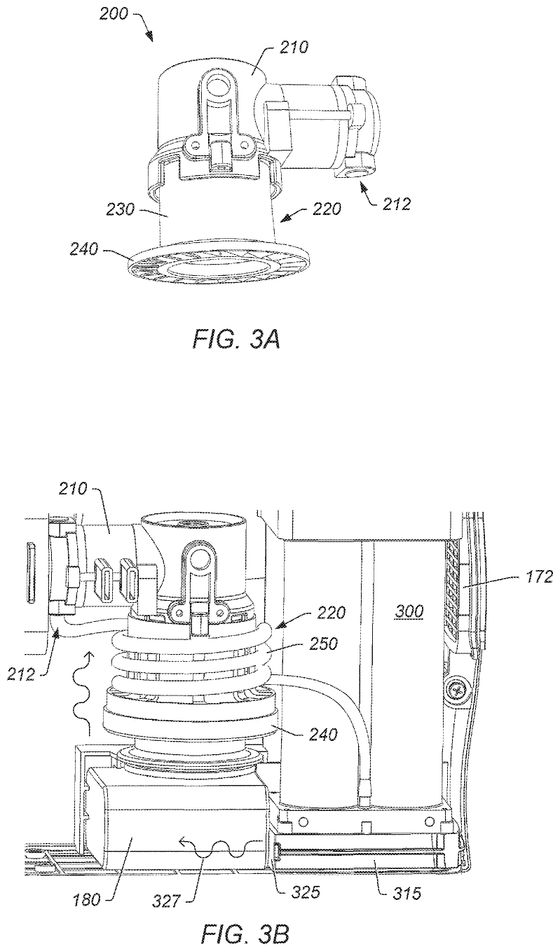

In some embodiments, compression system 200 includes one or more compressors. In another embodiment, compression system 200 includes a single compressor, coupled to all of the canisters of canister system 300. Turning to FIGS. 3A and 3B, a compression system 200 is depicted that includes compressor 210 and motor 220. Motor 220 is coupled to compressor 210 and provides an operating force to the compressor to operate the compression mechanism. For example, motor 220 may be a motor providing a rotating component that causes cyclical motion of a component of the compressor that compresses air. When compressor 210 is a piston type compressor, motor 220 provides an operating force which causes the piston of compressor 210 to be reciprocated. Reciprocation of the piston causes compressed air to be produced by compressor 210. The flow rate of the compressed air is, in part, estimated by the speed that the compressor is operated at (e.g., how fast the piston is reciprocated). Motor 220, therefore, may be a variable speed motor that is operable at various speeds to dynamically control the flow rate of air produced by compressor 210.

In one embodiment, compressor 210 includes a single head wobble type compressor having a piston. Other types of compressors may be used such as diaphragm compressors and other types of piston compressors. Motor 220 may be a DC or AC motor and provides the operating power to the compressing component of compressor 210. Motor 220, in an embodiment, may be a brushless DC motor. Motor 220 may be a variable speed motor capable of operating the compressing component of compressor 210 at variable speeds. Motor 220 may be coupled to controller 400, as depicted in FIG. 1, which sends operating signals to the motor to control the operation of the motor. For example, controller 400 may send signals to motor 220 to: turn the motor on, turn motor the off, and set the operating speed of the motor.

Compression system 200 inherently creates substantial heat. Heat is caused by the consumption of power by motor 220 and the conversion of power into mechanical motion. Compressor 210 generates heat due to the increased resistance to movement of the compressor components by the air being compressed. Heat is also inherently generated due to adiabatic compression of the air by compressor 210. Thus the continual pressurization of air produces heat in the enclosure. Additionally, power supply 180 may produce heat as power is supplied to compression system 200. Furthermore, users of the oxygen concentrator may operate the device in unconditioned environments (e.g., outdoors) at potentially higher ambient temperatures than indoors, thus the incoming air will already be in a heated state.

Heat produced inside oxygen concentrator 100 can be problematic. Lithium ion batteries are generally employed as a power supplies for oxygen concentrators due to their long life and light weight. Lithium ion battery packs, however, are dangerous at elevated temperatures and safety controls are employed in oxygen concentrator 100 to shut down the system if dangerously high power supply temperatures are detected. Additionally, as the internal temperature of oxygen concentrator 100 increases, the fraction of oxygen in the oxygen enriched gas generated by the concentrator may decrease. This is due, in part, to the decreasing amount of oxygen in a given volume of air at higher temperatures. If the fraction of oxygen drops below a predetermined amount, the oxygen concentrator 100 may automatically shut down, or sound an alarm.

Because of the compact nature of oxygen concentrators, dissipation of heat can be difficult. Solutions typically involve the use of one or more fans to create a flow of cooling air through the enclosure. Such solutions, however, require additional power from the power supply 180 and thus shorten the portable usage time of the oxygen concentrator. In an embodiment, a passive cooling system may be used that takes advantage of the mechanical power produced by motor 220 of the compressor 210. Referring to FIGS. 3A and 3B, compression system 200 includes motor 220 having an external rotating armature 230. Specifically, armature 230 of motor 220 (e.g. a DC motor) is wrapped around the stationary field that is driving the armature. Since motor 220 is a large contributor of heat to the overall system it is helpful to transfer heat off the motor and sweep it out of the enclosure. With the external high speed rotation, the relative velocity of the major component of the motor and the air in which it exists is very high. The surface area of the armature is larger if externally mounted than if it is internally mounted. Since the rate of heat exchange is proportional to the surface area and the square of the velocity, using a larger surface area armature mounted externally increases the ability of heat to be dissipated from motor 220. The gain in cooling efficiency by mounting the armature externally, allows the elimination of one or more cooling fans, thus reducing the weight and power consumption while maintaining the interior of the oxygen concentrator within the appropriate temperature range. Additionally, the rotation of the externally mounted armature creates movement of air proximate to the motor to create additional cooling.

Moreover, an external rotating armature may help the efficiency of the motor, allowing less heat to be generated. A motor having an external armature operates similar to the way a flywheel works in an internal combustion engine. When the motor is driving the compressor, the resistance to rotation is low at low pressures. When the pressure of the compressed air is higher, the resistance to rotation of the motor is higher. As a result, the motor does not maintain consistent ideal rotational stability, but instead surges and slows down depending on the pressure demands of the compressor. This tendency of the motor to surge and then slow down is inefficient and therefore generates heat. Use of an external armature adds greater angular momentum to the motor which helps to compensate for the variable resistance experienced by the motor. Since the motor does not have to work as hard, the heat produced by the motor may be reduced.

In an embodiment, cooling efficiency may be further increased by coupling an air transfer device 240 to external rotating armature 230. In an embodiment, air transfer device 240 is coupled to the external armature 230 such that rotation of the external armature causes the air transfer device 240 to create an airflow that passes over at least a portion of the motor. In an embodiment, air transfer device 240 includes one or more fan blades coupled to the armature. In an embodiment, a plurality of fan blades may be arranged in an annular ring such that the air transfer device 240 acts as an impeller that is rotated by movement of the external rotating armature. As depicted in FIGS. 3A and 3B, air transfer device 240 may be mounted to an outer surface of the external armature 230, in alignment with the motor. The mounting of the air transfer device 240 to the armature allows airflow to be directed toward the main portion of the external rotating armature, providing a cooling effect during use. In an embodiment, the air transfer device 240 directs air flow such that a majority of the external rotating armature is in the air flow path.

Further, referring to FIGS. 3A and 3B, air pressurized by compressor 210 exits compressor 210 at compressor outlet 212. A compressor outlet conduit 250 is coupled to compressor outlet 212 to transfer the compressed air to canister system 300. As noted previously, compression of air causes an increase in the temperature of the air. This increase in temperature can be detrimental to the efficiency of the oxygen concentrator. In order to reduce the temperature of the pressurized air, compressor outlet conduit 250 is placed in the air flow path produced by air transfer device 240. At least a portion of compressor outlet conduit 250 may be positioned proximate to motor 220. Thus, airflow, created by air transfer device 240, may contact both motor 220 and compressor outlet conduit 250. In one embodiment, a majority of compressor outlet conduit 250 is positioned proximate to motor 220. In an embodiment, the compressor outlet conduit 250 is coiled around motor 220, as depicted in FIG. 3B.

In an embodiment, the compressor outlet conduit 250 is composed of a heat exchange metal. Heat exchange metals include, but are not limited to, aluminum, carbon steel, stainless steel, titanium, copper, copper-nickel alloys or other alloys formed from combinations of these metals. Thus, compressor outlet conduit 250 can act as a heat exchanger to remove heat that is inherently caused by compression of the air. By removing heat from the compressed air, the number of molecules in a given volume at a given pressure is increased. As a result, the amount of oxygen enriched gas that can be generated by each canister during each pressure swing cycle may be increased.

The heat dissipation mechanisms described herein are either passive or make use of elements required for the oxygen concentrator 100. Thus, for example, dissipation of heat may be increased without using systems that require additional power. By not requiring additional power, the run-time of the battery packs may be increased and the size and weight of the oxygen concentrator may be minimized. Likewise, use of an additional box fan or cooling unit may be eliminated. Eliminating such additional features reduces the weight and power consumption of the oxygen concentrator.

As discussed above, adiabatic compression of air causes the air temperature to increase. During venting of a canister in canister system 300, the pressure of the gas being released from the canisters decreases. The adiabatic decompression of the gas in the canister causes the temperature of the gas to drop as it is vented. In an embodiment, the cooled vented gases from canister system 300 are directed toward power supply 180 and toward compression system 200. In an embodiment, base 315 of compression system 200 receives the vented gases from the canisters. The vented gases 327 are directed through base 315 toward outlet 325 of the base and toward power supply 180. The vented gases, as noted, are cooled due to decompression of the gases and therefore passively provide cooling to the power supply 180. When the compression system is operated, the air transfer device 240 will gather the cooled vented gases and direct the gases toward the motor of compression system 200. Fan 172 may also assist in directing the vented gas across compression system 200 and out of the housing 170. In this manner, additional cooling may be obtained without requiring any further power from the battery.

### Outlet System

An outlet system, coupled to one or more of the canisters, includes one or more conduits for providing oxygen enriched gas to a user. In an embodiment, oxygen enriched gas produced in either of canisters 302 and 304 is collected in accumulator 106 through check valves 142 and 144, respectively, as depicted schematically in FIG. 1. The oxygen enriched gas leaving the canisters may be collected in an oxygen accumulator 106 prior to being provided to a user. In some embodiments, a tube may be coupled to the accumulator 106 to provide the oxygen enriched gas to the user. Oxygen enriched gas may be provided to the user through an airway delivery device that transfers the oxygen enriched gas to the user's mouth and/or nose. In an embodiment, an outlet may include a tube that directs the oxygen toward a user's nose and/or mouth that may not be directly coupled to the user's nose.

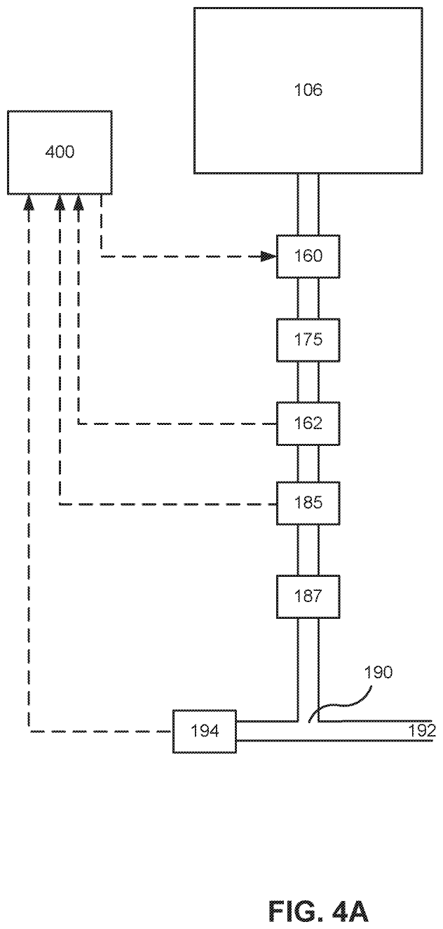

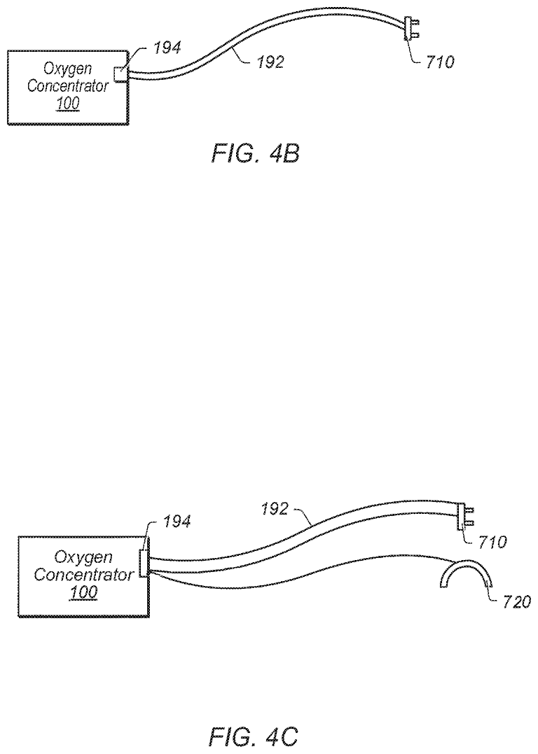

Turning to FIG. 4A, a schematic diagram of an embodiment of an outlet system for an oxygen concentrator is shown. A supply valve 160 may be coupled to an outlet tube to control the release of the oxygen enriched gas from accumulator 106 to the user. In an embodiment, supply valve 160 is an electromagnetically actuated plunger valve. Supply valve 160 is actuated by controller 400 to control the release of oxygen enriched gas to the user. Supply valve 160 is generally closed by default except as described below.

Oxygen enriched gas in accumulator 106 passes through supply valve 160 via flow restrictor 175 into oxygen sensor 162 as depicted in FIG. 4A. In an embodiment, oxygen sensor 162 may include one or more devices for determining an oxygen concentration of gas passing through the chamber. Oxygen enriched gas then passes through mass flow sensor 185 and particulate filter 187.

Actuation of supply valve 160 might not be timed or synchronized to the pressure swing adsorption process. Instead, actuation of supply valve 160 is, in some embodiments, synchronized to the user's breathing. Additionally, supply valve 160 may have continuously-valued actuation as opposed to on/off or binary actuation.

The flow restrictor 175 is a passive device configured to limit the flow rate of oxygen enriched gas being released to the user from accumulator 106 when supply valve 160 is actuated. The combination of supply valve 160 and flow restrictor 175 limits the amplitude of the delivered bolus even if supply valve 160 is binary. Alternatively, appropriate actuation of a continuously-actuatable supply valve 160 allows the controller 400 to establish a clinically effective and/or comfortable variable-amplitude profile for the bolus without the need for the flow restrictor 175.

Mass flow sensor 185 may be any sensor, or sensors, capable of estimating the mass flow rate of gas flowing through the conduit. Particulate filter 187 may filter bacteria, dust, granule particles, etc. prior to delivery of the oxygen enriched gas to the user. The oxygen enriched gas passes through filter 187 to connector 190 which sends the oxygen enriched gas to the user via conduit 192 and to pressure sensor 194.

The fluid dynamics of the outlet pathway, coupled with the actuation of supply valve 160, may result in a bolus of oxygen being delivered at the correct time and with an amplitude profile that assures rapid delivery into the user's lungs without excessive waste.

Oxygen sensor 162 may be used to determine an oxygen concentration of gas passing through the sensor. The oxygen sensor 162 may be a chemical oxygen sensor, an ultrasonic oxygen sensor, or some other type of oxygen sensor.

Mass flow sensor 185 may be used to determine the mass flow rate of gas flowing through the outlet system. Mass flow sensor 185 may be coupled to controller 400. The mass flow rate of gas flowing through the outlet system may be an indication of the breathing volume of the user. Changes in the mass flow rate of gas flowing through the outlet system may also be used to determine a breathing rate of the user. Controller 400 may control actuation of supply valve 160 based on the breathing rate and/or breathing volume of the user, as estimated by mass flow sensor 185.

Oxygen enriched gas passes through mass flow sensor 185 to filter 187. Filter 187 removes bacteria, dust, granule particles, etc. prior to providing the oxygen enriched gas to the user. The oxygen enriched gas passes through filter 187 to connector 190. Connector 190 may be a “Y” connector coupling the outlet of filter 187 to pressure sensor 194 and outlet conduit 192. Pressure sensor 194 may be used to monitor the pressure of the gas passing through conduit 192 to the user. Changes in pressure, sensed by pressure sensor 194, may be used to determine the onset(s) of inhalation (also referred to as trigger instant(s)). Hence, a pressure signal from such a pressure sensor may represent a breathing rate of a user. Controller 400 may control actuation of supply valve 160 based on the breathing rate and/or onset of inhalation of the user, as estimated by pressure sensor 194. In an embodiment, controller 400 may control actuation of supply valve 160 based on information provided by oxygen sensor 162, mass flow sensor 185, and pressure sensor 194.

Oxygen enriched gas may be provided to a user through conduit 192. In an embodiment, conduit 192 may be a silicone tube. Conduit 192 may be coupled to a user using an airway coupling member (e.g., airway delivery device 710), as depicted in FIGS. 4B and 4C. Airway delivery device 710 may be any device capable of providing the oxygen enriched gas to nasal cavities or oral cavities. Examples of airway coupling members include, but are not limited to: nasal masks, nasal pillows, nasal prongs, nasal cannulas, and mouthpieces. A nasal cannula airway delivery device is depicted in FIG. 4B. During use, oxygen enriched gas from oxygen concentrator 100 is provided to the user through conduit 192 and airway coupling member (e.g., airway delivery device 710). Airway delivery device 710 is positioned proximate to a user's airway (e.g., proximate to the user's mouth and or nose) to allow delivery of the oxygen enriched gas to the user while allowing the user to breathe air from the surroundings.

In an alternate embodiment, a mouthpiece may be used to provide oxygen enriched gas to the user. As shown in FIG. 4C, a mouthpiece 720 may be coupled to oxygen concentrator 100. Mouthpiece 720 may be the only device used to provide oxygen enriched gas to the user, or a mouthpiece may be used in combination with a nasal delivery device (e.g., a nasal cannula). As depicted in FIG. 4C, oxygen enriched gas may be provided to a user through both a nasal coupling member (e.g., airway delivery device 710) and a mouthpiece 720.

Mouthpiece 720 is removably positionable in a user's mouth. In one embodiment, mouthpiece 720 is removably couplable to one or more teeth in a user's mouth. During use, oxygen enriched gas is directed into the user's mouth via the mouthpiece. Mouthpiece 720 may be a night guard mouthpiece which is molded to conform to the user's teeth. Alternatively, mouthpiece may be a mandibular repositioning device. In an embodiment, at least a majority of the mouthpiece is positioned in a user's mouth during use.

During use, oxygen enriched gas may be directed to mouthpiece 720 when a change in pressure is detected proximate to the mouthpiece. In one embodiment, mouthpiece 720 may be coupled to a pressure sensor 194. When a user inhales air through the user's mouth, pressure sensor may detect a drop in pressure proximate to the mouthpiece. Controller 400 of oxygen concentrator 100 may deliver a bolus of oxygen enriched gas to the user at the onset of inhalation.

During typical breathing of an individual, inhalation may occur through the nose, through the mouth or through both the nose and the mouth. Furthermore, breathing may change from one passageway to another depending on a variety of factors. For example, during more active activities, a user may switch from breathing through their nose to breathing through their mouth, or breathing through their mouth and nose. A system that relies on a single mode of delivery (either nasal or oral), may not function properly if breathing through the monitored pathway is stopped. For example, if a nasal cannula is used to provide oxygen enriched gas to the user, an inhalation sensor (e.g., a pressure sensor or flow rate sensor) may be coupled to the nasal cannula to determine the onset of inhalation. If the user stops breathing through their nose, and switches to breathing through their mouth, the oxygen concentrator 100 may not know when to provide the oxygen enriched gas since there is no feedback from the nasal cannula. Under such circumstances, oxygen concentrator 100 may increase the flow rate and/or increase the frequency of providing oxygen enriched gas until the inhalation sensor detects an inhalation by the user. If the user switches between breathing modes often, the default mode of providing oxygen enriched gas may cause the oxygen concentrator 100 to work harder, potentially limiting the portable usage time of the system.

In an embodiment, a mouthpiece 720 is used in combination with an airway delivery device 710 (e.g., a nasal cannula) to provide oxygen enriched gas to a user, as depicted in FIG. 4C. Both mouthpiece 720 and airway delivery device 710 are coupled to an inhalation sensor. In one embodiment, mouthpiece 720 and airway delivery device 710 are coupled to the same inhalation sensor. In an alternate embodiment, mouthpiece 720 and airway delivery device 710 are coupled to different inhalation sensors. In either embodiment, inhalation sensor(s) may now detect the onset of inhalation from either the mouth or the nose. Oxygen concentrator 100 may be configured to provide oxygen enriched gas to the device (i.e. mouthpiece 720 or airway delivery device 710) proximate to which the onset of inhalation was detected. Alternatively, oxygen enriched gas may be provided to both mouthpiece 720 and the airway delivery device 710 if onset of inhalation is detected proximate either device. The use of a dual delivery system, such as depicted in FIG. 4C may be particularly useful for users when they are sleeping and may switch between nose breathing and mouth breathing without conscious effort.

### Controller System

Operation of oxygen concentrator 100 may be performed automatically using an internal controller 400 coupled to various components of the oxygen concentrator 100, as described herein. Controller 400 includes one or more processors 410 and internal memory 420, as depicted in FIG. 1. Methods used to operate and monitor oxygen concentrator 100 may be implemented by program instructions stored in memory 420 or a carrier medium coupled to controller 400, and executed by one or more processors 410. A memory medium may include any of various types of memory devices or storage devices. The term “memory medium” is intended to include an installation medium, e.g., a Compact Disc Read Only Memory (CD-ROM), floppy disks, or tape device; a computer system memory or random access memory such as Dynamic Random Access Memory (DRAM), Double Data Rate Random Access Memory (DDR RAM), Static Random Access Memory (SRAM), Extended Data Out Random Access Memory (EDO RAM), Rambus Random Access Memory (RAM), etc.; or a non-volatile memory such as a magnetic media, e.g., a hard drive, flash memory, or optical storage. The memory medium may comprise other types of memory as well, or combinations thereof.

In some embodiments, controller 400 includes processor 410 that includes, for example, one or more field programmable gate arrays (FPGAs), microcontrollers, etc. included on a circuit board disposed in oxygen concentrator 100. Processor 410 is capable of executing programming instructions stored in memory 420. In some embodiments, programming instructions may be built into processor 410 such that a memory external to the processor may not be separately accessed (i.e., the memory 420 may be internal to the processor 410).

Processor 410 may be coupled to various components of oxygen concentrator 100, including, but not limited to compression system 200, one or more of the valves used to control fluid flow through the system (e.g., valves 122, 124, 132, 134, 152, 154, 160), oxygen sensor 162, pressure sensor 194, mass flow sensor 185, temperature sensor, cooling fans, humidity sensor, actigraphy sensor, altimeter, and any other component that may be electrically controlled or monitored. In some embodiments, a separate processor (and/or memory) may be coupled to one or more of the components.

Controller 400 is programmed to operate oxygen concentrator 100 and is further programmed to monitor the oxygen concentrator 100 for malfunction states. For example, in one embodiment, controller 400 is programmed to trigger an alarm if the system is operating and no breathing is detected by the user for a predetermined amount of time. For example, if controller 400 does not detect a breath for a period of 75 seconds, an alarm LED may be lit and/or an audible alarm may be sounded. If the user has truly stopped breathing, for example, during a sleep apnea episode, the alarm may be sufficient to awaken the user, causing the user to resume breathing. The action of breathing may be sufficient for controller 400 to reset this alarm function. Alternatively, if the system is accidently left on when output conduit 192 is removed from the user, the alarm may serve as a reminder for the user to turn oxygen concentrator 100 off to conserve power.

Controller 400 is further coupled to oxygen sensor 162, and may be programmed for continuous or periodic monitoring of the oxygen concentration of the oxygen enriched gas passing through oxygen sensor 162. A minimum oxygen concentration threshold may be programmed into controller 400, such that the controller lights an LED visual alarm and/or an audible alarm to warn the user of the low concentration of oxygen.

Controller 400 is also coupled to internal power supply 180 and is capable of monitoring the level of charge of the internal power supply. A minimum voltage and/or current threshold may be programmed into controller 400, such that the controller lights an LED visual alarm and/or an audible alarm to warn the user of low power condition. The alarms may be activated intermittently and at an increasing frequency as the battery approaches zero usable charge.

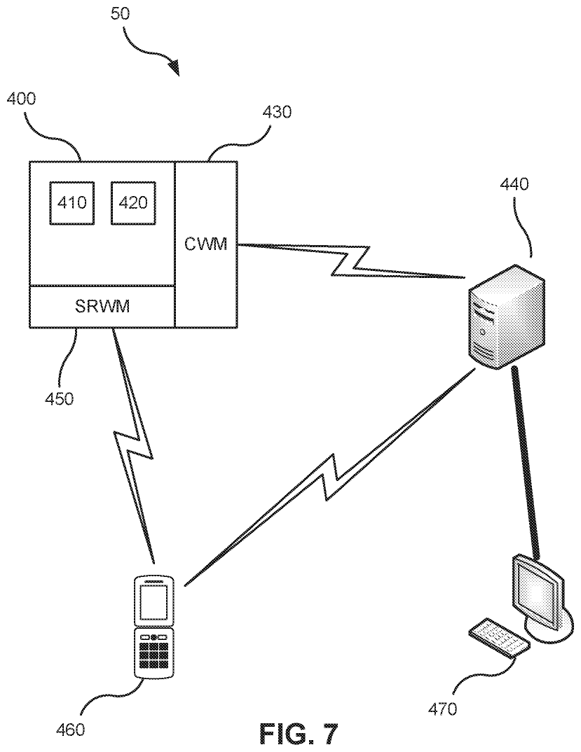

Controller 400 may be communicatively coupled to one or more external computing devices to make up a connected oxygen therapy system. FIG. 7 illustrates one implementation of a connected oxygen therapy system 50, in which the controller 400 may include a Cellular Wireless Module (CWM) 430, or other wireless communications module, configured to allow the controller 400 to communicate, using a wireless communication protocol such as the Global System for Mobile Telephony (GSM) or other protocol (e.g., WIFI), with a remote computing device (e.g., server 440) such as a cloud-based server such as over a network. The controller 400 may also include a short range wireless module (SRWM) 450 configured to enable the controller 400 to communicate, using a short range wireless communication protocol such a Bluetooth, with a portable computing device 460 such as a smartphone. The portable computing device 460 (e.g., smartphone) may be associated with a user of the POC 100.

The server 440 may also be in wireless communication with the portable computing device 460 using a wireless communication protocol such as GSM. A processor of the portable computing 460 (e.g., smartphone) may execute a program known as an “app” to control the interaction of the smartphone with the POC 100 and/or the server 440.

The server 440 may also be in communication with a personal computing device 470 via a wired or wireless connection to a wide-area network such as the Internet, or a local-area network such as an Ethernet. A processor of the personal computing device 470 may execute a “client” program to control the interaction of the personal computing device 470 with the server 440. One example of a client program is a browser.

A connected oxygen therapy system 50 may comprise a plurality or “fleet” of POCs (not shown) like the POC 100, all in communication with the server 440, either directly or via respective portable computing devices 460 associated with respective users of the POCs. The personal computing device 470 may be associated with a health management entity (HME) that is responsible for the therapy of a population of users of the fleet of POCs.

Further functions of controller 400 are described in detail in other sections of this disclosure.

### Outer Housing—Control Panel

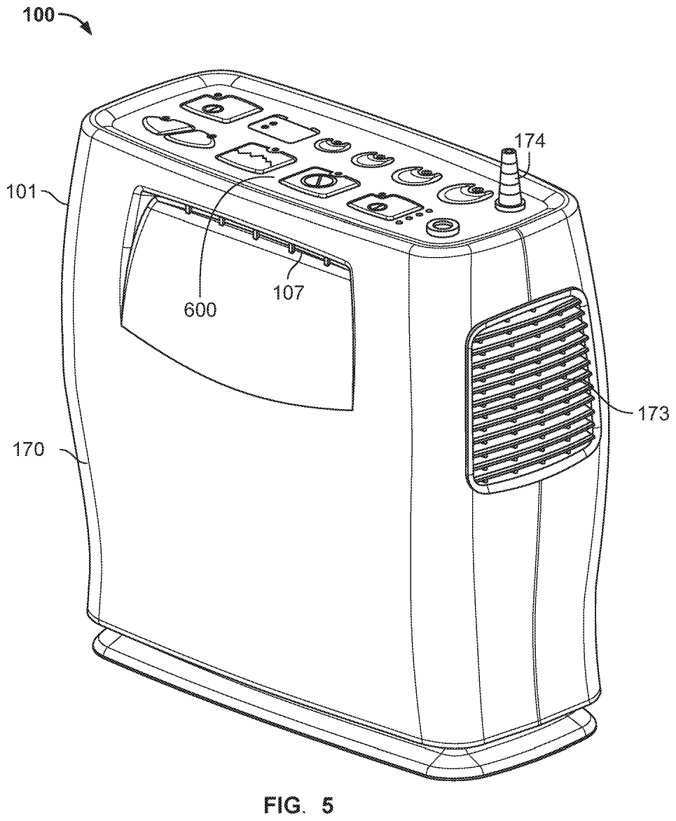

FIG. 5 depicts an embodiment of an outer housing 170 of an oxygen concentrator 100. In some embodiments, outer housing 170 may be comprised of a light-weight plastic. Outer housing includes compression system inlets 107, cooling system passive inlet 101 and outlet 173 at each end of outer housing 170, outlet port 174, and control panel 600. Inlet 101 and outlet 173 allow cooling air to enter the housing, flow through the housing, and exit the interior of housing 170 to aid in cooling of the oxygen concentrator 100. Compression system inlets 107 allow air to enter the compression system. Outlet port 174 is used to attach a conduit to provide oxygen enriched gas produced by the oxygen concentrator 100 to a user.

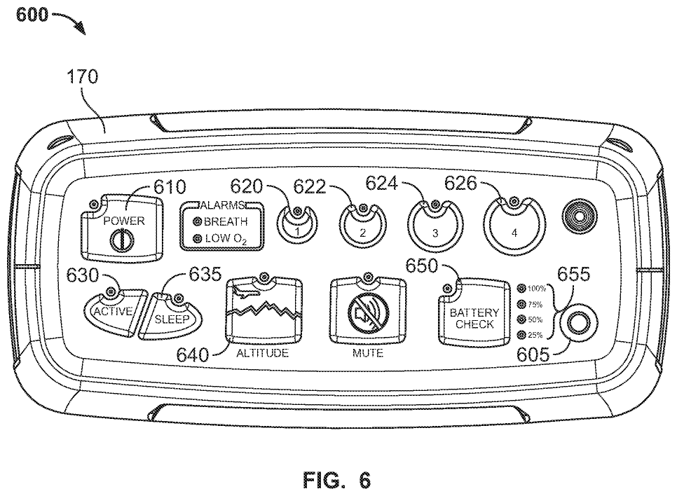

Control panel 600 serves as an interface between a user and controller 400 to allow the user to initiate predetermined operation modes of the oxygen concentrator 100 and to monitor the status of the system. Charging input port 605 may be disposed in control panel 600. FIG. 6 depicts an embodiment of control panel 600.

In some embodiments, control panel 600 may include buttons to activate various operation modes for the oxygen concentrator 100. For example, control panel may include power button 610, dosage buttons 620 to 626, active mode button 630, sleep mode button 635, and a battery check button 650. In some embodiments, one or more of the buttons may have a respective LED that may illuminate when the respective button is pressed (and may power off when the respective button is pressed again). Power button 610 may power the system on or off. If the power button is activated to turn the system off, controller 400 may initiate a shutdown sequence to place the system in a shutdown state (e.g., a state in which both canisters are pressurized). Dosage buttons 620, 622, 624, and 626 allow the prescribed continuous flow rate of oxygen enriched gas to be selected (e.g., 1 LPM by button 620, 2 LPM by button 622, 3 LPM by button 624, and 4 LPM by button 626). Altitude button 640 may be selected when a user is going to be in a location at a higher elevation than the oxygen concentrator 100 is regularly used by the user. The adjustments made by the oxygen concentrator 100 in response to activating altitude mode are described in more detail herein.

Battery check button 650 initiates a battery check routine in the oxygen concentrator 100 which results in one or more relative battery power remaining LEDs 655 being illuminated on control panel 600.

A user may have a low breathing rate or depth if relatively inactive (e.g., asleep, sitting, etc.) as estimated by comparing the detected breathing rate or depth to a threshold. The user may have a high breathing rate or depth if relatively active (e.g., walking, exercising, etc.). An active/sleep mode may be estimated automatically and/or the user may manually indicate a respective active or sleep mode by pressing button 630 for active mode and button 635 for sleep mode. The adjustments made by the oxygen concentrator 100 in response to activating active mode or sleep mode are described in more detail herein.

### Methods of Delivery of Oxygen Enriched Gas

The main use of an oxygen concentrator 100 is to provide supplemental oxygen to a user. One or more flow rate settings may be selected on a control panel 600 of the oxygen concentrator 100, which then will control operations to achieve production of the oxygen enriched gas according to the selected flow rate setting. In some versions, a plurality of flow rate settings may be implemented (e.g., five flow rate settings). As described in more detail herein, the controller may implement a POD (pulsed oxygen delivery) or demand mode of operation to regulate size of one or more released boluses to achieve delivery of the oxygen enriched gas according to the selected flow rate setting.

In order to maximise the effect of the delivered oxygen enriched gas, controller 400 may be programmed to synchronise release of each bolus of the oxygen enriched gas with the user's inhalations. Releasing a bolus of oxygen enriched gas to the user as the user inhales may prevent wastage of oxygen by not releasing oxygen, for example, when the user is exhaling. For concentrators that operate in POD mode, the flow rate settings on the control panel 600 may correspond to minute volumes (bolus volume multiplied by breathing rate per minute) of delivered oxygen, e.g. 0.2 LPM, 0.4 LPM, 0.6 LPM, 0.8 LPM, 1.1 LPM.

Oxygen enriched gas produced by oxygen concentrator 100 is stored in an oxygen accumulator 106 and released to the user as the user inhales. The amount of oxygen enriched gas provided by the oxygen concentrator 100 is controlled, in part, by supply valve 160. In an embodiment, supply valve 160 is opened for a sufficient amount of time to provide the appropriate amount of oxygen enriched gas, as estimated by controller 400, to the user. In order to minimize the wastage of oxygen, the oxygen enriched gas may be delivered as a bolus in synchrony with the user's detected inhalation. For example, the bolus of oxygen enriched gas may be delivered in the first few milliseconds of a user's inhalation.

In an embodiment, pressure sensor 194 may be used to determine the onset of inhalation by the user. For example, the user's inhalation may be detected by using pressure sensor 194. In use, a conduit for providing oxygen enriched gas is coupled to a user's nose and/or mouth through the airway delivery device 710 and/or 720. At the onset of an inhalation, the user begins to draw air into their body through the nose and/or mouth. As the air is drawn in, a negative pressure is generated at the end of the conduit, due, in part, to the venturi action of the air being drawn across the end of the delivery conduit. Such a drop in pressure may be detected in the signal provided by the pressure sensor 194 or other suitable sensor, to indicate the onset of inhalation. Upon detection of the onset of inhalation, supply valve 160 is opened to deliver a bolus of oxygen enriched gas from the accumulator 106. In some cases, opening of the supply valve may optionally be delayed in relation to the onset detection by implementation of a waiting interval known as the onset delay. A positive change or rise in the pressure indicates an exhalation by the user and is generally a time that release of oxygen enriched gas is discontinued. Generally when a positive pressure change is sensed, supply valve 160 is closed until the next onset of inhalation. Alternatively, supply valve 160 may be closed after a predetermined interval known as the bolus duration. By measuring the intervals between adjacent onsets of inhalation, the user's breathing rate may be estimated. By measuring the intervals between onsets of inhalation and the following onsets of exhalation, the user's inspiratory time may be estimated.

### Estimation of Sieve Bed Remaining Capacity

At the start of a PSA cycle for either canister (302 or 304), feed gas is fed in to one of the canisters to increase the pressure of the canister by a predetermined canister pressure rise ΔP (the “pressurization phase”). The pressurization phase duration may be predetermined as the time taken to increase the canister pressure of a fresh canister by the canister pressure rise ΔP. After the pressurization phase, the check valve 142 or 144 opens and the “adsorption phase” commences during which the canister pressure stabilises or rises more slowly than during the pressurization phase. After another predetermined interval the canister is depressurized by closing the inlet valve 122 or 124 and opening the outlet valve 132 or 134 for another predetermined interval (the “depressurization phase”).

As a sieve bed becomes deactivated, less nitrogen is adsorbed by the adsorbent, and therefore more of the nitrogen is available to increase the pressure inside the sieve bed. The pressure therefore increases more rapidly as a given amount of feed gas is fed in. The time required for the sieve bed pressure to increase by the canister pressure rise ΔP for a given input flow rate thus reflects the remaining adsorption capacity of the adsorbent material in the sieve bed, assuming no change in ambient conditions.

There are typically two void spaces within a packed sieve bed. One is the void space between particles, which is the volume of the canister not occupied by the solid matter of the particles. This void space is called bed void. The other void space is within each particle as the adsorbent used in portable oxygen concentrator is porous. This void space is called particle void. The combination of these two void spaces compose the total void in a packed bed. The void volumes (bed void and particle void, which summed together may be considered the total void) are usually stated as a fraction, ε.

Bed void fraction ε_{b} is the ratio of bed void volume to canister volume. The bed void fraction ε_{b} can be calculated as

$${ɛ}_{b} = \frac{{ρ}_{bulk}}{{ρ}_{particle}} \qquad ( 1 )$$

where ρ_{bulk} is the “bulk” density of the adsorbent in the canister (mass per unit volume) and ρ_{particle} is the “matter” density of the individual adsorbent particles (mass per unit volume, with the same units as ρ_{bulk}). The bulk density ρ_{bulk} is the ratio of the mass of adsorbent particles in a canister (a known canister parameter) to the volume V of the canister, typically 500 to 800 milligrams per cubic centimeter. The matter density ρ_{particle} is a quantity specified by the manufacturer of the adsorbent material, typically 900 to 1500 milligrams per cubic centimeter.

If ρ_{particle} is not known or not supplied by the manufacturer, then ε_{b} can be calculated from sieve bed pressure drop through the Ergun equation, known by those skilled in the art.

Particle void fraction or εₚ is the ratio of particle void volume to particle volume. This value may be provided by the material manufacturer. If this value is not available through the material manufacturer, it can be computed through gas pycnometer data. A gas pycnometer can provide material skeletal density ρₛₖₑₗₑₜₐₗ which is mass to volume of the solid component in a particle (taking out all the void and empty spaces). Using this data and ρ_{particle}, the particle void fraction εₚ can be calculated as

$${ɛ}_{p} = 1 - \frac{{ρ}_{particle}}{{ρ}_{skeletal}} \qquad ( 2 )$$

Based on both εₚ and ε_{b}, a total void fraction εₜₒₜₐₗ can be calculated as

$${ɛ}_{total} = {ɛ}_{b} + {ɛ}_{p} - ( {ɛ}_{b} \times {ɛ}_{p} ) \qquad ( 3 )$$

When feed gas molecules are fed into the sieve bed, they either stay in the void (in which case they increase the pressure in the canister) or are adsorbed by the adsorbent (in which case they don't). If the pressure in the canister increases by ΔP, then the number n_{void} of moles of gas that have increased the pressure, i.e. stayed in the void, may be computed from the ideal gas equation as follows:

$${n}_{void} = \frac{Δ {PV}_{void}}{RT} \qquad ( 4 )$$

where R is the universal gas constant (approximately equal to 8.31 in SI units), Tis the temperature of the feed gas within the sieve bed, and V_{void} is the void volume, i.e. the total void fraction εₜₒₜₐₗ of equations (3) times the volume V of the canister. The canister pressure rise ΔP is a predetermined parameter of the PSA cycle. The temperature T of the gas within the sieve bed may be set to the temperature of the feed gas measured by one or more temperature sensors located in the gas path in the vicinity of the canister, or more accurately, a number of temperature sensors located along the sieve bed/s. For example, a temperature sensor may be located between the compressor 200 and one or both canisters, and/or within one or both canisters, and/or at the outlet of one or both canisters. In the case of multiple temperature sensors, the value of T may be estimated as a combination of multiple temperature measurements from the respective temperature sensors, e.g. an average value.

The total number N of moles of feed gas fed into the sieve bed (which is made up of the n_{void} moles of equation (4) that stayed in the void and the n_{ads} moles that were adsorbed by the adsorbent) may be computed from the mass flow rate Q (in moles per second) of the compression system 200 and the pressure rise time Δt, i.e. the time taken to increase the canister pressure by the canister pressure rise ΔP, as follows:

$$N = Q Δ t \qquad ( 5 )$$

A value for the mass flow rate Q of feed gas being fed into the sieve bed by the compression system 200 may be obtained in a number of ways. One way is to use a mass flow sensor at the output of the compressor 200 to give a real-time-accurate measurement of Q. Another way is to use a function that calculates the mass flow rate Q from the current compressor characteristics, e.g. motor speed, and current ambient conditions, such as one or more of temperature, barometric pressure, altitude, and humidity. Such a function may be developed during calibration of compressor 200 and embodied in, for example, a look-up table, such as a multi-dimensional look-up table, stored in memory 420 at the time of manufacture of the POC 100. Alternatively, such a function may be developed (for a reciprocating compressor 210) from the ideal gas law. Ambient conditions not available due to the absence of appropriate sensors may be set to typical values such as 20° C. for temperature, 70% for relative humidity, and sea level for altitude.

The mass flow rate Q of feed gas may vary with time over the pressurization phase. In such implementations, the total number N of moles of feed gas fed into the sieve bed may be computed as the integral of the mass flow rate Q(t) with respect to time over the pressure rise time Δt.

If the canister contains a layer of desiccant upstream of the adsorbent, then the number N of moles of gas actually reaching the sieve bed is less than the number of moles of gas leaving the compressor. In such implementations, the value N in the following formulae may be discounted from the value computed by equation (5). The amount of discount is a function of the molar fraction of water vapour in the feed gas, which in turn is a function of the relative ambient humidity H, and the ambient temperature and pressure. The relative humidity H may be converted to the partial pressure of water vapour in the feed gas using well known methods, e.g. the Antoine equation, based on the ambient temperature. The partial pressure of water vapour in the feed gas may then be divided by the ambient pressure to obtain the molar fraction of water vapour in the feed gas. The molar fraction of water vapour may then be discounted by some small amount to reflect the fact that the desiccant layer does not adsorb all the water vapour in the feed gas. The value of N computed by equation (5) may then be multiplied by one minus the discounted molar fraction of water vapour.

In other implementations, the void fraction of the desiccant material may be estimated from manufacturers' data in similar fashion to the void fraction εₜₒₜₐₗ of the adsorbent material as described earlier. A mass-weighted average of the void fractions of desiccant material and adsorbent may be used as an overall void fraction to calculate the void volume Goo for use in equation (4).

The “unadsorbed fraction” X of the moles of feed gas fed into the sieve bed may be computed as

$$X = \frac{{n}_{void}}{N} \qquad ( 6 )$$

where N is the number of moles fed into the sieve bed.

The fraction X is a measure of the exhaustion of the sieve bed, in that when the sieve bed is fully exhausted, the sieve bed will have a fraction X equal to one, as no input gas molecules will be adsorbed, while the fraction X will be lower for a fresh sieve bed. The remaining capacity of the sieve bed is inversely related to the fraction X, in the sense that as the capacity decreases over the sieve bed life, the value of X increases.

An alternative implementation takes into account the fact that oxygen is not adsorbed at the same rate as nitrogen by the adsorbent bed. This effect may be modelled by assuming that the oxygen molecules fed into the sieve bed (21% of the feed gas) are adsorbed specifically at the top part of the sieve bed, then, as the nitrogen adsorption front is progressing through the bed, released into the void to increase the pressure inside the canister. So in addition to the fraction X of unadsorbed molecules times the total number N of moles of feed gas fed into the sieve bed, a further 21% of the remaining (1−X)N moles of feed gas also contribute to the canister pressure rise. That is,

$${n}_{void} = XN + 0.21 ( 1 - X ) N \qquad ( 7 )$$

Equation (7) may be rearranged to allow the fraction X to be computed as

$$X = \frac{{n}_{void} - 0.21 N}{0.79 N} \qquad ( 8 )$$

Note that when the sieve bed is fully exhausted and when all the gas feed molecules go to the void to increase the pressure (n_{void}=QΔt), the fraction X evaluates to one under Equation (8), just as under the simpler Equation (6).

When the sieve bed is fresh (i.e. remaining capacity C=100%), the fraction X will have some value X_{fresh} that is less than one. For any given sieve bed, this “fresh fraction” X_{fresh} is an operational characteristic of the sieve bed that may either be estimated from the amount and properties of the adsorbent material in the canister, the parameters of the PSA process, and the ambient conditions, or computed during one or more initial pressurizations of a sieve bed of the same type (i.e. same dimensions and contents) from the compressor mass flow rate Q, the gas temperature T, the “fresh” pressure rise time Δt_{fresh}, the canister pressure rise ΔP, and the void volume V_{void}, using Equation (4) and Equation (6) or Equation (8). If multiple initial pressurizations are used, a value for Δt_{fresh} (also an operational characteristic of the sieve bed) may be estimated as a combination, e.g. an average, of the pressure rise times measured at each pressurization.

Then during sieve bed use, the fraction X may be computed from the compressor mass flow rate Q, the gas temperature T, the pressure rise time Δt, the canister pressure rise ΔP, and the void volume V_{void} using Equation (4) and either Equation (6) or Equation (8). The remaining capacity C of the sieve bed may then be estimated as a function of the fraction X using the “fresh” value of 1 (when X=X_{fresh}) and the “exhausted” value of 0 (when X equals the “exhausted” fraction, namely one). In one implementation the function may be a linear interpolation:

$$C = \frac{1 - X}{1 - {X}_{fresh}} \qquad ( 9 )$$

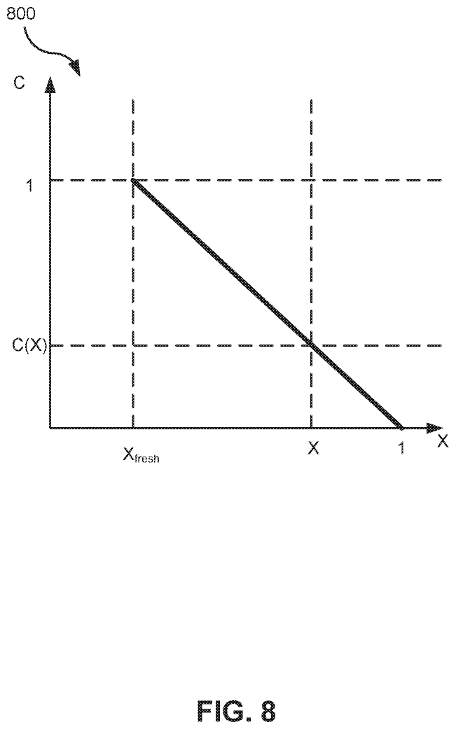

FIG. 8 is a graph 800 illustrating the linear interpolation of remaining capacity C(X) from the computed fraction X based on the fresh fraction X_{fresh}.

An alternative implementation is based on the observation from Equation (6) that, given constant gas temperature T, canister pressure rise ΔP, void volume V_{void}, and compressor mass flow rate Q, the pressure rise time Δt is inversely proportional to the fraction X Under the alternative formulation in Equation (8), the pressure rise time Δt is not inversely proportional to the fraction X, but X falls monotonically as Δt rises, so it may be said that the pressure rise time Δt is generally inverse to the fraction X.

The interpolation (e.g., linear) of the capacity C may therefore be based on the pressure rise time Δt rather than the fraction X To do this, a fresh pressure rise time Δt_{fresh} and an “exhausted” pressure rise time Δtₑₓ (another operational characteristic of the sieve bed) may be estimated from the fresh fraction X_{fresh} and the exhausted fraction (unity) using equations (4) to (6) or measured from a fresh and an exhausted sample of a given sieve bed type (dimensions and contents) respectively. The remaining capacity C of a sieve bed of the same type may be estimated during usage as a function of the measured pressure rise time Δt using the fresh pressure rise time Δt_{fresh} and the exhausted pressure rise time Δtₑₓ. In one implementation, the function may be a linear interpolation:

$$C = \frac{Δ t - Δ {t}_{ex}}{Δ {t}_{fresh} - Δ {t}_{ex}} \qquad ( 10 )$$

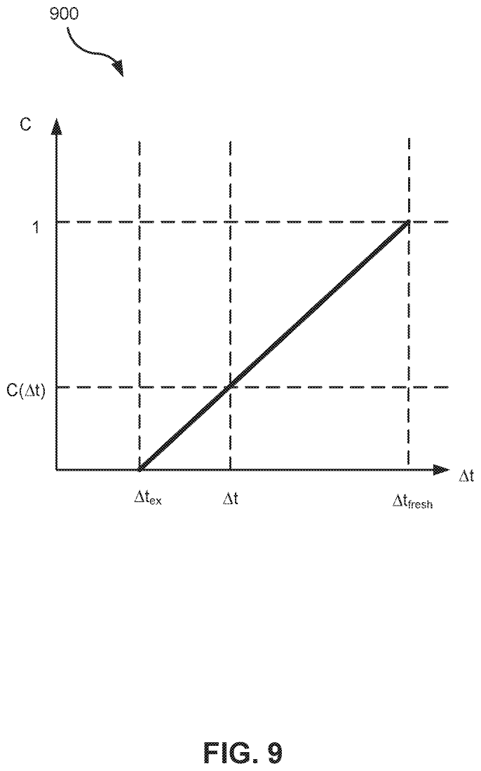

FIG. 9 is a graph 900 illustrating an example of the linear interpolation of remaining capacity C(Δt) from a measured pressure rise time Δt based on the fresh pressure rise time Δt_{fresh} and the exhausted pressure rise time Δtₑₓ.

The pressure rise time Δt taken by a given compressor to achieve a given canister pressure rise ΔP for a sieve bed with a given void volume at a given remaining capacity will vary with ambient conditions (one or more of temperature, humidity, and pressure). Therefore, for greater accuracy in estimation of remaining capacity C, the pressure rise time Δt may be adjusted before applying Equation (10) to compensate for any differences between the current, in-use ambient conditions and those prevailing at the time of measurement of the fresh pressure rise time Δt_{fresh} and the exhausted pressure rise time Δtₑₓ. In some implementations, the ambient conditions may be measured using appropriate sensors such as temperature, humidity, and pressure sensors at the time when the fresh pressure rise time Δt_{fresh} and the exhausted pressure rise time Δtₑₓ were measured. These measurements may be stored in memory 420. The same sensors may be used to measure current ambient conditions during usage, and the value of the pressure rise time Δt may be adjusted based on any change in ambient conditions before applying Equation (10). Alternatively, the fresh pressure rise time Δt_{fresh} and the exhausted pressure rise time Δtₑₓ may be adjusted (leaving the pressure rise time Δt unadjusted) based on any change in ambient conditions since those parameters were measured. In one example implementation of adjustment, a measured pressure rise time Δt may be scaled in inverse proportion to the change in absolute temperature. In another example implementation, a measured pressure rise time Δt may be scaled in inverse proportion to the change in compressor mass flow rate Q. (The change in compressor mass flow rate Q may be modelled using a function that relates the mass flow rate Q to the current ambient conditions, as described above.)

Any one or more of the aforementioned equations, and any form of their operations, may be implemented as one or more functions by a device, such as a POC, for estimating remaining capacity of a sieve bed. Such function(s) may, in some cases, be implemented with one or more data look-up tables.

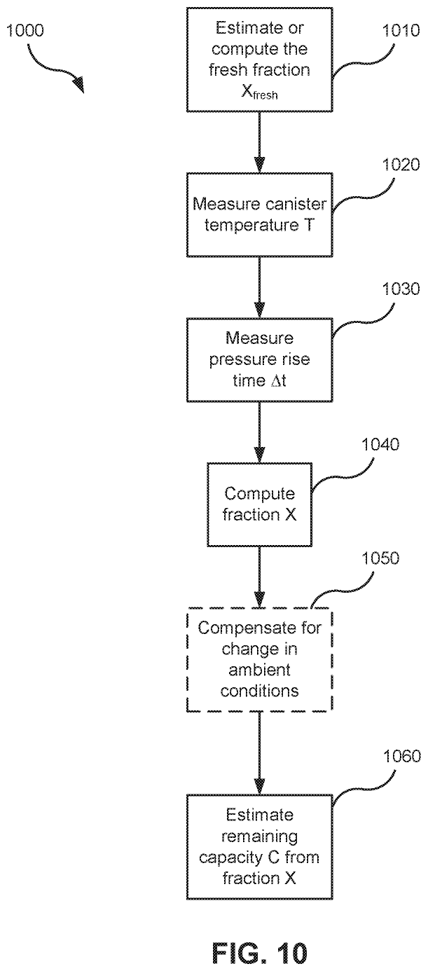

For example, FIG. 10 contains an example flowchart of a method 1000 of estimating the remaining capacity C of a sieve bed in one implementation of the present technology. The method 1000 may be executed by the one or more processors, such as the one or more processors 410 of the controller 400, configured by program instructions, such as including, as previously described, the one or more functions and/or associated data corresponding thereto, stored in a memory such as the memory 420 of the POC 100. Alternatively, some or all of the steps of the method 1000 may be similarly executed by one or more processors of an external computing device with which the controller 400 of the POC 100 is configured to communicate, as described above. In this latter implementation, the processors 410 may be configured by program instructions stored in the memory 420 of the POC 100 to transmit to the external computing device the measurements and parameters necessary for the performance of those steps that are to be carried out at the external computing device. The external computing device may be the remote computing device (e.g., server 440), the portable computing device 460, or the personal computing device 470.

Optionally, the method 1000 may start at step 1010, which estimates or computes the fresh fraction X_{fresh}. The fresh fraction X_{fresh} may be computed as a function of the compressor mass flow rate Q, the gas temperature T, the fresh pressure rise time Δ_{fresh}, the canister pressure rise ΔP, and the void volume V_{void} using Equation (6) or Equation (8) above. Step 1010 may be an initial step that may be carried out once for all sieve beds of the same type, or once for a given sieve bed. For example, such information may be predetermined (e.g., by other devices or by the POC 100) and stored within a memory of a POC 100 or other device for use with the latter steps of the method. The subsequent steps 1020 to 1050 may be carried out every time the remaining capacity of a sieve bed of the same type as used in step 1010, or the same sieve bed as used in step 1010, is to be estimated.

Step 1020 measures the temperature T of the canister being pressurized in a pressurization phase of a PSA cycle. Step 1030, such as simultaneously with step 1020, measures the pressure rise time Δt. Step 1040 then computes the fraction X as a function of the compressor mass flow rate Q, the gas temperature T, the pressure rise time Δt, the canister pressure rise ΔP, and the void volume V_{void} using Equation (6) or Equation (8) above. An optional step 1050 adjusts the fraction X or the fresh fraction X_{fresh} based on the change in ambient conditions since the fresh fraction X_{fresh} was measured, as described above. Finally, step 1060 computes an estimate of the remaining capacity C as a function of the fraction X and the fresh fraction X_{fresh} using Equation (9).

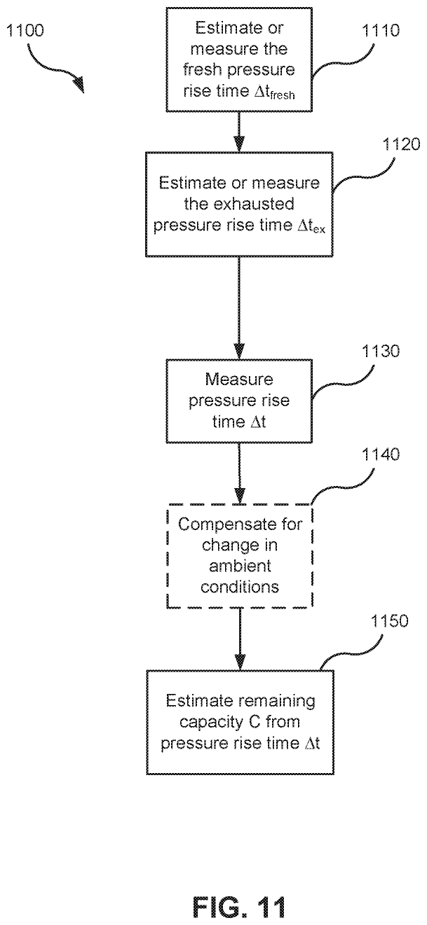

FIG. 11 contains a flowchart of a method 1100 of estimating the remaining capacity C of a sieve bed in one implementation of the present technology. The method 1100 may be executed by the controller 400 configured by program instructions stored in the memory 420 of the POC 100. Alternatively, some or all of the steps of the method 1100 may be executed by one or more processors of an external computing device with which the controller 400 of the POC 100 is configured to communicate, as described above. In this latter implementation, the processors 410 may be configured by program instructions stored in the memory 420 of the POC 100 to transmit to the external computing device the measurements and parameters necessary for the performance of those steps that are to be carried out at the external computing device. The external computing device may be the remote computing device (e.g., server 440) the portable computing device 460, or the personal computing device 470.

The method 1100 may optionally start at step 1110, which estimates or measures the fresh pressure rise time Δt_{fresh} of a fresh sieve bed. Step 1120 estimates or measures the exhausted pressure rise time Δtₑₓ of the same sieve bed when exhausted, or an exhausted sieve bed of the same type.

Steps 1110 and 1120 are optional initial steps that may be carried out once for all sieve beds of the same type. For example, such information may be predetermined (e.g., by other devices or by the POC 100) and stored within a memory of a POC 100 or other device for use with the latter steps of the method. Step 1110 may also be carried out once for a given sieve bed, such as in a setup process. The subsequent steps 1130 to 1150 may be carried out every time the remaining capacity of a sieve bed of the same type as was used in step 1110 and 1120, or the same sieve bed that was used for step 1110, is to be estimated.

Step 1130 measures the pressure rise time Δt of a pressurization phase of a PSA cycle. An optional step 1140 adjusts the pressure rise time Δt, or the fresh pressure rise time Δt_{fresh} and the exhausted pressure rise time Δtₑₓ, based on the change in ambient conditions since the fresh pressure rise time Δt_{fresh} and the exhausted pressure rise time Δtₑₓ were measured, as described above. Step 1150 then computes an estimate of the remaining capacity C as a function of the pressure rise time Δt, the fresh pressure rise time Δt_{fresh}, and the exhausted pressure rise time Δtₑₓ using Equation (10).

Further alternative methods may use other parameters of the pressurization phase of the pressure-time characteristic P(t) of the sieve bed in analogous fashion to how the methods 1000 and 1100 use the pressure rise time Δt to estimate the sieve bed remaining capacity C. An example of such a parameter is the initial slope dP/dt of the pressure-time characteristic, which is low when the remaining capacity C is 100% and increases as the remaining capacity C decreases. Another example of such a parameter is the slope dP/dt of the pressure-time characteristic during the adsorption phase. Yet another example of such a parameter is the ratio of the initial slope dP/dt of the pressure-time characteristic to the slope dP/dt of the pressure-time characteristic during the adsorption phase.

Yet further alternative methods use parameters of the depressurization phase of the pressure-time characteristic P(t) of the sieve bed in analogous fashion to how the methods 1000 and 1100 use the pressure rise time Δt of the pressurization phase to estimate the sieve bed remaining capacity C. An example of such a parameter is the pressure fall time.

As mentioned above, controller 400 is coupled to oxygen sensor 162, and may be programmed for continuous or periodic monitoring of the oxygen concentration of the oxygen enriched gas passing through oxygen sensor 162. The oxygen concentration pO₂ has been used to produce an estimate Cₒₓ of sieve bed remaining capacity. The remaining capacity Cₒₓ estimated from the oxygen concentration may be compared with the remaining capacity C estimated from the pressure-time characteristic parameters as described above. A significant difference between the two estimates of remaining capacity may be used to indicate an internal system fault within the POC 100 such as leak, condensation, sensor fault or gas blockage.

Alternatively, the oxygen concentration pO₂ may be used as a “sanity check” on the remaining capacity C estimated from the pressure-time characteristic. For example, if the remaining capacity C is approaching 0% but the oxygen concentration pO₂ remains near its maximum value, this may serve as an indicator of a system fault. Conversely, if the oxygen concentration pO₂ has fallen significantly (e.g. below 90%), this may serve as an indicator to confirm the declining estimate of remaining capacity.

Multiple estimates of remaining capacity C(t₁), C(t₂), . . . C(t_{N}) at times t₁, t₂, t_{N} may be converted to an estimate R of remaining usage time before sieve bed exhaustion. In one implementation, a trend or time profile C(t) may be extracted from the estimates C(t₁), C(t₂), C(t_{N}) of remaining capacity, and the time profile C(t) may be extrapolated to estimate the time to at which the remaining capacity C(t₀) will reach zero, assuming the continuance of a similar usage pattern that give rise to the estimates C(t₁), C(t₂), . . . C(t_{N}). The estimate R of remaining usage time may then be set to the difference between to and the current time.

### Use of the Remaining Capacity/Usage Time Estimate

The estimates of remaining sieve bed capacity and/or usage time may be further utilised by the various entities in a connected oxygen therapy system 50.

In one implementation, the remaining capacity and/or usage time estimate may be displayed on the control panel 600 of the POC 100. For example, the LEDs 655 may be used to indicate the current value of the remaining capacity estimate (e.g. 100%, 75%, 50%, 25% as illustrated) rather than remaining battery power. This display may occur in response to activation of a separate button (not shown) on the control panel 600. Similarly, a numeric (e.g. 8-segment) display (not shown) could be used to display the current value of the remaining capacity and/or usage time estimate.

In another implementation, the “app” running on the portable computing device 460 could cause the value of the remaining capacity and/or usage time estimate to be displayed on a display of the portable computing device 460. This could occur on the instruction of the server 440 via a “push notification” to the app, or on the initiative of the app itself. Optionally, in some cases, the processor of the portable computing device may access data measured by the POC, such as by receiving such data from the POC, and compute the value of the remaining capacity and/or usage time estimate using any of the processing methodologies as previously described.

In a further implementation, the server 440 may be configured to host a portal system. The portal system may receive, from the portable computing device 460 or directly from the POC 100, data relating to the operation of the POC 100. For example, such operational data may include estimates of remaining capacity or usage time of sieve beds in a POC 100 or the measurements for computing such estimates at a server of the portal system. As described above, the personal computing device 470 may execute a client application such as a browser to allow a user of the personal computing device 470 (such as a representative of an HME) to access the operational data of the POC 100, and other POCs in the connected oxygen therapy system 50, via the portal system hosted by the server 440. In this fashion, such a portal system may be utilised by an HME to manage a population of users of POC devices, e.g. the POC device 100, in the connected oxygen therapy system 50.

The portal system may provide actionable insights into user or device condition for the population of POC devices and their users based on the operational data received by the portal system. Such insights may be based on rules that are applied to the operational data. In one implementation, the estimated remaining usage times of a fleet of POCs may be displayed to a representative of an HME on a display of a personal computing device 470 in a “window” of a client program interacting with the portal system. Further, a rule may be applied to each remaining usage time estimate. One example of such a rule is “If the remaining usage time for a POC is less than three weeks, highlight the POC in the display of usage times”. Application of such a rule to the estimated remaining usage times results in the highlighting on the display of POCs with sieve beds approaching exhaustion. The highlighted POCs may then be noted by the HME for imminent sieve bed replacement. This is one example of the kind of rule-based fleet management made possible by the methods 1000 and 1100 of estimating sieve bed remaining capacity operating within the connected oxygen therapy system 50.

Optionally, such as in case where the POC 100 determines an estimate of the remaining capacity C of a sieve bed, the POC 100 may communicate a message, which may be based on the estimate, such as by a comparison with a threshold (e.g., if the estimate is at or below a threshold), to an external computing device of the system 50 such as to provide a notification message of a need for a sieve bed. Such a message may comprise a request for a new sieve bed such as for arranging a purchase or replacement order for a new sieve bed via an ordering or fulfillment system implemented with any of the devices of FIG. 7. Such a message may also be generated by any of the devices of the system 50 that receives either the estimate or the measurements and parameters necessary for determining the estimate. In such a case, the message may be further transmitted to other systems, such as a purchasing, ordering or fulfillment system or server(s) that may be configured to communicate with a device of the system 50 for arranging and/or completing such orders. Still further, in some versions, the POC may make a change in a control parameter of the POC based on the estimate or a comparison of the estimate and one or more thresholds. For example, one or more parameters for control of the PSA cycle of the POC may be adjusted based on the comparison. Such adjustments may include, for example, parameters for the various valve timings of the valves that control flow through the canisters for feed and purge cycles and/or compressor speed, etc. Such adjustments may be implemented for increasing remaining sieve bed usage life if a partially impaired bed is detected (e.g., less than 100%, 50% etc.) or resuming normal operating parameters for a detection of a renewed bed (e.g., greater than 50% or at or near 100%). Optionally, any of the devices of the system 50 may be configured to communicate command(s) to the POC for the POC to implement a change in a control parameter(s) of the POC, such as when such devices detect a need for such a change in the POC operation based on the estimate or a comparison of the estimate and one or more thresholds.

### General Remarks

In the present disclosure, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the technology may be apparent to those skilled in the art in view of this description. Accordingly, this description is to be construed as illustrative only and is for the purpose of teaching those skilled in the art the general manner of carrying out the technology. It is to be understood that the forms of the technology shown and described herein are to be taken as embodiments. Elements and materials may be substituted for those illustrated and described herein, parts and processes may be reversed, and certain features of the technology may be utilized independently, all as would be apparent to one skilled in the art after having the benefit of this description of the technology. Changes may be made in the elements described herein without departing from the spirit and scope of the technology as described in the following claims.

For example, the aforementioned technology may be further considered with respect to the examples of the following descriptive paragraphs:

EXAMPLE 1. A method of estimating a remaining capacity of a sieve bed in an oxygen concentrator, the method comprising:

measuring a pressure-time characteristic of the sieve bed for a phase of a pressure swing adsorption cycle of the oxygen concentrator;

extracting a parameter of the pressure-time characteristic; and

estimating the remaining capacity using the parameter of the pressure-time characteristic.

EXAMPLE 2. The method of EXAMPLE 1, wherein the estimating further uses a fresh value of the parameter for a fresh sieve bed of a same type as the sieve bed.

EXAMPLE 3. The method of EXAMPLE 2, further comprising adjusting at least one of (a) the parameter and (b) the fresh value of the parameter to compensate for a change in ambient conditions since the fresh value of the parameter was measured.

EXAMPLE 4. The method of EXAMPLE 2, wherein the one or more functions use an exhausted value of the parameter for an exhausted sieve bed of a same type as the sieve bed.

EXAMPLE 5. The method of EXAMPLE 4, further comprising adjusting the exhausted value of the parameter to compensate for a change in ambient conditions since the exhausted value of the parameter was measured.

EXAMPLE 6. The method of any one of EXAMPLES 2 to 5, wherein the estimating the remaining capacity comprises interpolating between the parameter and the fresh value of the parameter.

EXAMPLE 7. The method of any one of EXAMPLES 1 to 6, wherein the parameter is an unadsorbed fraction of feed gas fed into the sieve bed.

EXAMPLE 8. The method of EXAMPLE 7, wherein extracting the parameter comprises dividing an amount of unadsorbed feed gas by an amount of feed gas fed into the sieve bed.

EXAMPLE 9. The method of any one of EXAMPLES 1 to 6, wherein the estimating further uses a value of the parameter for an exhausted sieve bed of the same type as the sieve bed.

EXAMPLE 10. The method of EXAMPLE 9, wherein the parameter is a pressure rise time.

EXAMPLE 11. The method of any one of EXAMPLES 1 to 10, further comprising:

repeating the measuring, extracting, and estimating to obtain a further estimate of remaining capacity, and

estimating remaining usage time of the sieve bed from the estimate and the further estimate of remaining capacity.

EXAMPLE 12. The method of any one of EXAMPLES 1 to 11, further comprising displaying, on a display of the oxygen concentrator, an indicator of the estimate of remaining capacity.

EXAMPLE 13. An oxygen concentrator comprising:

a sieve bed containing a gas separation adsorbent;

a compression system configured to feed a feed gas into the sieve bed;

a memory; and

a controller comprising one or more processors, the one or more processors configured by program instructions stored in the memory to execute the method of estimating the remaining capacity of the sieve bed of any one of EXAMPLES 1 to 12.

EXAMPLE 14. A connected oxygen therapy system comprising:

a portable oxygen concentrator comprising a sieve bed containing a gas separation adsorbent;

an external computing device in communication with the portable oxygen concentrator;

a memory, and

a processor configured by program instructions stored in the memory to estimate a remaining capacity of the sieve bed, the processor configured to:

measure a pressure-time characteristic of the sieve bed for a phase of a pressure swing adsorption cycle of the oxygen concentrator;

extract a parameter of the pressure-time characteristic; and

estimate the remaining capacity using the parameter of the pressure-time characteristic.

EXAMPLE 15. The connected oxygen therapy system of EXAMPLE 14, wherein the processor and the memory are part of the portable oxygen concentrator.

EXAMPLE 16. The connected oxygen therapy system of EXAMPLE 15, wherein the processor is further configured to transmit the remaining capacity estimate to the external computing device.

EXAMPLE 17. The connected oxygen therapy system of EXAMPLE 14, wherein the processor and the memory are part of the external computing device.

EXAMPLE 18. The connected oxygen therapy system of any one of EXAMPLES 14 to 17, further comprising a display.

EXAMPLE 19. The connected oxygen therapy system of EXAMPLE 18, wherein the processor is further configured to display an indicator of the remaining capacity that is estimated on the display.

EXAMPLE 20. The connected oxygen therapy system of any one of EXAMPLES 14 to 19, wherein the external computing device is a portable computing device.

EXAMPLE 21. The connected oxygen therapy system of any one of EXAMPLES 14 to 19, wherein the external computing device is a server.

EXAMPLE 22. The connected oxygen therapy system of EXAMPLE 21, further comprising a personal computing device in communication with the server.

EXAMPLE 23. The connected oxygen therapy system of EXAMPLE 22, wherein the personal computing device is configured to interact with a portal system hosted by the server.

EXAMPLE 24. The connected oxygen therapy system of EXAMPLE 23, wherein the personal computing device is configured to:

receive the remaining capacity estimate from the portal system; and

display the remaining capacity estimate on a display of the personal computing device.

EXAMPLE 25. The connected oxygen therapy system of EXAMPLE 21, further comprising a portable computing device in communication with the server.

EXAMPLE 26. The connected oxygen therapy system of EXAMPLE 25, wherein the portable computing device is configured to:

receive the remaining capacity estimate from the server; and

display the remaining capacity estimate on a display of the portable computing device.

EXAMPLE 27. Apparatus comprising:

means for measuring a pressure-time characteristic of a sieve bed for a phase of a pressure swing adsorption cycle of the oxygen concentrator;

means for extracting a parameter of the pressure-time characteristic; and

means for estimating a remaining capacity of the sieve bed using the parameter of the pressure-time characteristic.

Label list

| | |
|---|---|
| oxygen therapy system | 50 |
| oxygen concentrator | 100 |
| inlets | 101 |
| accumulator | 106 |
| inlet | 107 |
| inlet muffler | 108 |
| inlet valve | 122 |
| inlet valve | 124 |
| outlet | 130 |
| outlet valve | 132 |
| muffler | 133 |
| outlet valve | 134 |
| check valve | 142 |
| check valve | 144 |
| flow restrictor | 151 |
| valve | 152 |
| flow restrictor | 153 |
| valve | 154 |
| flow restrictor | 155 |
| supply valve | 160 |
| oxygen sensor | 162 |
| outer housing | 170 |
| fan | 172 |
| Outlet | 173 |
| outlet port | 174 |
| flow restrictor | 175 |
| power supply | 180 |
| mass flow sensor | 185 |
| filter | 187 |
| connector | 190 |
| conduit | 192 |
| pressure sensor | 194 |
| compression system | 200 |
| compressor | 210 |
| compressor outlet | 212 |
| motor | 220 |
| external rotating armature | 230 |
| air transfer device | 240 |
| compressor outlet conduit | 250 |
| canister system | 300 |
| canister | 302 |
| canister | 304 |
| base | 315 |
| outlet | 325 |
| gases | 327 |
| controller | 400 |
| Processor(s) | 410 |
| memory | 420 |
| cellular wireless module | 430 |
| remote computing device | 440 |
| short range wireless module | 450 |
| portable computing device | 460 |
| personal computing device | 470 |
| control panel | 600 |
| input port | 605 |
| power button | 610 |
| button | 620 |
| button | 622 |
| button | 624 |
| button | 626 |
| button | 630 |
| button | 635 |
| altitude button | 640 |
| battery check button | 650 |
| LEDs | 655 |
| airway coupling member | 710 |
| mouthpiece | 720 |
| graph | 800 |
| graph | 900 |
| method | 1000 |
| step | 1010 |
| step | 1020 |
| step | 1030 |
| step | 1040 |
| step | 1050 |
| step | 1060 |
| method | 1100 |
| step | 1110 |
| step | 1120 |
| step | 1130 |
| step | 1140 |
| step | 1150 |

1. A method of estimating a remaining capacity of a sieve bed in an oxygen concentrator, the method comprising:
accessing a parameter of a measured pressure-time characteristic of the sieve bed for a phase of a pressure swing adsorption cycle of the oxygen concentrator;
accessing one or more functions of the parameter of the measured pressure-time characteristic and one or more operational characteristics of the sieve bed; and
estimating the remaining capacity by applying the one or more functions to the parameter of the measured pressure-time characteristic.

2. The method of claim 1, wherein the one or more functions use a fresh value of the parameter for a fresh sieve bed of a same type as the sieve bed.

3. The method of claim 2, further comprising adjusting at least one of (a) the fresh value of the parameter, and (b) the parameter, to compensate for a change in ambient conditions since the fresh value of the parameter was measured.

4. The method of any one of claims 2 to 3, wherein the one or more functions use an exhausted value of the parameter for an exhausted sieve bed of a same type as the sieve bed.

5. The method of claim 4, further comprising adjusting the exhausted value of the parameter to compensate for a change in ambient conditions since the exhausted value of the parameter was measured.

6. The method of any one of claims 4 to 5, wherein the one or more functions comprise an interpolation using the fresh value of the parameter and the exhausted value of the parameter.

7. The method of any one of claims 1 to 6 wherein the parameter is a pressure rise time.

8. The method of any one of claims 1 to 7, wherein the parameter is an unadsorbed fraction of feed gas fed into the sieve bed.

9. The method of claim 8 wherein the one or more functions comprise a parameter representing a total number of moles of feed gas fed into the sieve bed.

10. The method of any of any one of claims 8 to 9 wherein the one or more functions comprise a parameter representing a mass flow rate of feed gas fed into the sieve bed.

11. The method of any one of claims 8 to 10 wherein the one or more functions comprise a parameter representing an amount of unadsorbed feed gas fed into the sieve bed.

12. The method of any one of claims 8 to 11 wherein the one or more functions comprise parameters representing a change in pressure over the phase, a void volume of a canister of the sieve bed, a temperature of feed gas fed into the sieve bed, and a universal gas constant.

13. The method of any one of claims 8 to 12, wherein the one or more functions comprise dividing an amount of unadsorbed feed gas fed into the sieve bed by an amount of feed gas fed into the sieve bed.

14. The method of any one of claims 1 to 13 wherein the one or more functions comprise one or more look-up tables.

15. The method of any one of claims 1 to 14, further comprising:
repeating the accessing and the estimating to obtain a further estimate of remaining capacity, and
estimating a remaining usage time of the sieve bed from the estimate and the further estimate of remaining capacity.

16. The method of any one of claims 1 to 15, further comprising displaying, on a display of the oxygen concentrator, an indicator of the remaining capacity that is estimated.

17. The method of any one of claims 1 to 16, further comprising generating a message based on the remaining capacity that is estimated.

18. The method of any one of claims 1 to 17 further comprising measuring the pressure-time characteristic of the sieve bed for the phase of the pressure swing adsorption cycle of the oxygen concentrator to generate the parameter.

19. The method of claim 18 further comprising repeating the measuring to obtain a further estimate of remaining capacity.

20. A method of estimating a remaining capacity of a sieve bed in an oxygen concentrator, the method comprising:
measuring a pressure-time characteristic of the sieve bed for a phase of a pressure swing adsorption cycle of the oxygen concentrator;
extracting a parameter of the pressure-time characteristic; and
estimating the remaining capacity using the parameter of the pressure-time characteristic.

21. The method of claim 20, wherein the parameter is an unadsorbed fraction of feed gas fed into the sieve bed.

22. The method of any one of claims 20 to 21 wherein the parameter is a pressure rise time.

23. The method of any one of claims 20 to 22, further comprising:
repeating the measuring, extracting, and estimating to obtain a further estimate of remaining capacity, and
estimating remaining usage time of the sieve bed from the estimate and the further estimate of remaining capacity.

24. An oxygen concentrator comprising:
a sieve bed containing a gas separation adsorbent;
a compression system configured to feed a feed gas into the sieve bed;
a memory; and
a controller comprising one or more processors, the one or more processors configured by program instructions stored in the memory to execute the method of estimating remaining capacity of the sieve bed of any one of claims 1 to 23.

25. An oxygen concentrator comprising:
a sieve bed containing a gas separation adsorbent;
a compression system configured to feed a feed gas into the sieve bed;
a memory; and
a controller configured to:
access a parameter of a measured pressure-time characteristic of the sieve bed for a phase of a pressure swing adsorption cycle of the oxygen concentrator;
access one or more functions of the parameter of the measured pressure-time characteristic and one or more operational characteristics of the sieve bed; and
estimate a remaining capacity of the sieve bed by applying the one or more functions to the parameter of the measured pressure-time characteristic.

26. An oxygen concentrator comprising:
a sieve bed containing a gas separation adsorbent;
a compression system configured to feed a feed gas into the sieve bed;
a memory; and
a controller configured to:
measure a pressure-time characteristic of the sieve bed for a phase of a pressure swing adsorption cycle of the oxygen concentrator;
extract a parameter of the pressure-time characteristic; and
estimate a remaining capacity of the sieve bed using the parameter of the pressure-time characteristic.

27. A connected oxygen therapy system comprising:
a portable oxygen concentrator comprising a sieve bed containing a gas separation adsorbent;
an external computing device in communication with the portable oxygen concentrator;
a memory; and
a processor configured by program instructions stored in the memory to estimate a remaining capacity of the sieve bed, the processor configured to:
access a parameter of a measured pressure-time characteristic of the sieve bed for a phase of a pressure swing adsorption cycle of the portable oxygen concentrator;
access one or more functions of the parameter of the measured pressure-time characteristic and one or more operational characteristics of the sieve bed; and
estimate the remaining capacity by applying the one or more functions to the parameter of the measured pressure-time characteristic.

28. The connected oxygen therapy system of claim 27, wherein the processor and the memory are part of the portable oxygen concentrator.

29. The connected oxygen therapy system of claim 28, wherein the processor is further configured to transmit the remaining capacity estimate to the external computing device.

30. The connected oxygen therapy system of claim 27, wherein the processor and the memory are part of the external computing device.

31. The connected oxygen therapy system of any one of claims 27 to 30, further comprising a display.

32. The connected oxygen therapy system of claim 31, wherein the processor is further configured to display an indicator of the remaining capacity that is estimated on the display.

33. The connected oxygen therapy system of any one of claims 27 to 32, wherein the external computing device is a portable computing device.

34. The connected oxygen therapy system of any one of claims 27 to 32, wherein the external computing device is a server.

35. The connected oxygen therapy system of claim 34, further comprising a personal computing device in communication with the server.

36. The connected oxygen therapy system of claim 35, wherein the personal computing device is configured to interact with a portal system hosted by the server.

37. The connected oxygen therapy system of claim 36, wherein the personal computing device is configured to:
receive the remaining capacity estimate from the portal system; and
display the remaining capacity estimate on a display of the personal computing device.

38. The connected oxygen therapy system of claim 34, further comprising a portable computing device in communication with the server.

39. The connected oxygen therapy system of claim 38, wherein the portable computing device is configured to:
receive the remaining capacity estimate from the server; and
display the remaining capacity estimate on a display of the portable computing device.

40. Apparatus comprising:
means for accessing a parameter of a measured pressure-time characteristic of a sieve bed for a phase of a pressure swing adsorption cycle of an oxygen concentrator;
means for accessing one or more functions of the parameter of the measured pressure-time characteristic and one or more operational characteristics of the sieve bed; and
means for estimating a remaining capacity of the sieve bed by applying the one or more functions to the parameter of the measured pressure-time characteristic.

41. Apparatus comprising:
means for measuring a pressure-time characteristic of a sieve bed for a phase of a pressure swing adsorption cycle of an oxygen concentrator;
means for extracting a parameter of the pressure-time characteristic; and
means for estimating a remaining capacity of the sieve bed using the parameter of the pressure-time characteristic.
